(19) United States
(12) Patent Application Publication
(10) Pub. No.: US 2023/0184747 A1
(43) Pub. Date: Jun. 15, 2023

(54) T CELL-BASED METHODS FOR PREDICTING POLYPEPTIDE IMMUNOGENICITY

(71) Applicant: GENENTECH, INC., South San Francisco, CA (US)

(72) Inventors: Shan Chung, South San Francisco, CA (US); Sivan Cohen, South San Francisco, CA (US)

(73) Assignee: GENENTECH, INC., South San Francisco, CA (US)

(21) Appl. No.: 18/104,825

(22) Filed: Feb. 2, 2023

Related U.S. Application Data
(63) Continuation of application No. PCT/US2021/044704, filed on Aug. 5, 2021.
(60) Provisional application No. 63/215,199, filed on Jun. 25, 2021.
(60) Provisional application No. 63/062,991, filed on Aug. 7, 2020.

(51) Int. Cl. G01N 33/50; C12N 5/0783; G01N 33/68

(52) U.S. Cl. CPC G01N 33/5047; C12N 5/0636; G01N 33/6854

(57) ABSTRACT

The presently disclosed subject matter provides methods for determining the propensity of a composition, e.g., a composition comprising an antibody or a fragment thereof, to elicit production of anti-drug antibodies (ADAs) and kits for performing such methods.

### CROSS-REFERENCE TO RELATED APPLICATIONS

This application is a continuation of International Patent Application No. PCT/US2021/044704, filed Aug. 5, 2021, which claims priority to U.S. Provisional Application No. 63/062,991, filed Aug. 7, 2020, and U.S. Provisional Application No. 63/215,199, filed Jun. 25, 2021, the contents of each of which are incorporated by reference in their entireties, and to each of which priority is claimed.

### FIELD OF THE INVENTION

The present disclosure relates to methods for determining the propensity of a composition to elicit production of anti-drug antibodies (ADAs) and kits for performing such methods.

### BACKGROUND

Therapeutics (e.g., antibodies) have greatly improved the treatment of an increasing number of serious and difficult to treat diseases. Unfortunately, such therapeutics may elicit the production of anti-drug antibodies (ADAs) when administered to a patient. ADAs can have neutralizing effects on the therapeutic. These neutralizing effects can include limiting the activity of the therapeutic, increasing clearance of the therapeutic, and a potential reduction in overall clinical response attributable to administration of the therapeutic. In certain instances, the production of ADAs has also coincided with the occurrence of severe adverse events in patients, including hypersensitivity reactions and anaphylaxis.

Understanding the immunogenicity of a therapeutic in the preclinical phase of drug development can improve the likelihood of success of the therapeutic in subsequent clinical phases. While immunogenic epitopes are generally predicted using in silico tools, several cell-based techniques have been developed to determine the immunogenic potential of preclinical therapeutic candidates. One such technique is called major histocompatibility complex (MHC) class II-associated peptide proteomics (MAPPs). MAPPs involves incubating a population of antigen-presenting cells (APCs), e.g., dendritic cells, with a therapeutic of interest, e.g., a polypeptide-based therapeutic. The APCs will internalize and process the therapeutic into short peptides. The peptides are loaded onto MHC class II molecules and presented on the surface of the APC. Immunoprecipitating and analyzing these MHC-peptide complexes via liquid chromatography mass spectrometry (LC/MS) allows for identification of potentially immunogenic epitopes in the therapeutic. Another technique for determining the immunogenic potential of a preclinical therapeutic candidate is the T cell proliferation assay, which involves the detection of T cell proliferation after co-culture with APCs, e.g., dendritic cells, that have been incubated with the polypeptide-based therapeutic of interest. These techniques, however, are labor intensive, time consuming and require numerous pieces of high-cost equipment. Accordingly, there is a need in the art for a more time-efficient and cost-effective method for determining the propensity of a therapeutic, e.g., a polypeptide-based therapeutic, to elicit production of ADAs.

### SUMMARY

The present disclosure provides methods for determining the propensity of a composition to elicit the production of antibodies specific for the composition compared to a reference propensity. In certain embodiments, a method of the present disclosure can include (a) culturing lymphocytes in the presence of the composition to generate stimulated lymphocytes; (b) culturing lymphocytes in the absence of the composition to generate unstimulated lymphocytes; (c) determining the percentage of the stimulated lymphocytes that are CD4+ and express: (i) CD134; (ii) CD137; or (iii) CD134 and CD137; (d) determining the percentage of the unstimulated lymphocytes that are CD4+ and express: (i) CD134; (ii) CD137; or (iii) CD134 and CD137; and (e) calculating a stimulation index value. In certain embodiments, when the stimulation index value in (e) is greater than or equal to a reference stimulation index value then the composition has a greater propensity to elicit antibodies specific to said composition. In certain embodiments, when the stimulation index value in (e) is less than the reference stimulation index value then the composition has a lesser propensity to elicit antibodies specific to said composition. In certain embodiments, the stimulation index value can be determined by: (i) dividing the percentage of stimulated lymphocytes determined in (c) with the percentage of unstimulated lymphocytes determined in (d), (ii) outlier sum analysis and/or (iii) linear regression. In certain embodiments, the lymphocytes are obtained from a single donor. In certain embodiments, the lymphocytes are obtained from about 20 donors to about 50 donors, e.g., from about 35 to about 45 donors. In certain embodiments, the lymphocytes are obtained from at least about 20 donors, at least about 25 donors, at least about 30 donors, at least about 35 donors, at least about 40 donors or at least about 45 donors.

In certain embodiments, a method for determining the propensity of a composition to elicit the production of antibodies specific to the composition includes (a) separately culturing lymphocytes from individual donors in the presence of the composition to generate stimulated lymphocytes; (b) separately culturing lymphocytes from the individual donors in the absence of the composition to generate unstimulated lymphocytes; (c) determining the percentage of the stimulated lymphocytes from the individual donors that are CD4+ and express: (i) CD134; (ii) CD137; or (iii) CD134 and CD137; (d) determining the percentage of the unstimulated lymphocytes from the donors that are CD4+ and express: (i) CD134; (ii) CD137; or (iii) CD134 and CD137; (e) calculating a stimulation index value for each of the donors; and (f) calculating a number of reactive lymphocyte donors where the donors' stimulation index value is greater than or equal to a reference value stimulation index value and the number of unstimulated lymphocyte donors where the donors' stimulation index value is less than the reference stimulation index value. In certain embodiments, the stimulation index value can be determined by: (i) dividing the percentage of stimulated lymphocytes of an individual donor determined in (c) with the percentage of unstimulated lymphocytes of that individual donor determined in (d), (ii) outlier sum analysis and/or (iii) linear regression. In certain embodiments, the composition has a high propensity to elicit the production of antibodies specific to the composition if the number of stimulated donors is greater than 30% of the total number of donors. In certain embodiments, the composition has a low propensity to elicit the production of antibodies specific to the composition if the number of stimulated donors is less than 20% of the total number of donors. In certain embodiments, the lymphocytes are obtained from about 20 donors to about 50 donors, e.g., from about 35 to about 45 donors. In certain embodiments, the lymphocytes are obtained from at least about 20 donors, at least about 25 donors, at least about 30 donors, at least about 35 donors, at least about 40 donors or at least about 45 donors.

In certain embodiments, the composition comprises a neoantigen. In certain embodiments, the composition is a peptide, a polypeptide or a small molecule compound. In certain embodiments, the polypeptide is an antibody or fragment thereof, e.g., the antibody or fragment thereof is a human, humanized or chimeric antibody. In certain embodiments, the composition is an antibody-drug conjugate (ADC). In certain embodiments, the antibody or fragment thereof is a bispecific antibody.

The present disclosure further provides methods for determining the propensity of a neoantigen to elicit an immune response specific to the neoantigen relative to a reference antigen. For example, but not by way of limitation, the method can include (a) culturing lymphocytes in the presence of the neoantigen to generate stimulated lymphocytes; (b) culturing lymphocytes in the absence of the neoantigen to generate unstimulated lymphocytes; (c) determining the percentage of the stimulated lymphocytes that are CD4+ and express: (i) CD134; (ii) CD137; or (iii) CD134 and CD137; (d) determining the percentage of the unstimulated lymphocytes that are CD4+ and express: (i) CD134; (ii) CD137; or (iii) CD134 and CD137; and (e) calculating a stimulation index value. In certain embodiments, the stimulation index value can be determined by (i) dividing the percentage of stimulated lymphocytes determined in (c) with the percentage of unstimulated lymphocytes determined in (d), (ii) outlier sum analysis and/or (iii) by linear regression. In certain embodiments, when the stimulation index value in (e) is greater than or equal to a reference stimulation index value then the neoantigen has a greater propensity to elicit an immune response specific to the neoantigen and when the stimulation index value in (e) is less than the reference stimulation index value then the neoantigen has a lesser propensity to elicit an immune response specific to the neoantigen.

In certain embodiments, the reference stimulation index value is the stimulation index value of a reference composition, e.g., a composition that does not elicit production of ADAs in a clinical setting or has a low propensity to elicit production of ADAs in a clinical setting. In certain embodiments, the reference stimulation index value is from about 1.0 to about 4.0, i.e., from about 1.0 to about 2.0. In certain embodiments, the reference stimulation index value is about 1.6 or greater, about 1.7 or greater or about 1.8 or greater.

In certain embodiments, the lymphocytes comprise T cells. In certain embodiments, at least 30% of the lymphocytes comprise T cells. In certain embodiments, the T cells are CD8−. In certain embodiments, at least 10% of the T cells comprise CD8− T cells. In certain embodiments, about 1×10⁵ to about 1×10⁷ lymphocytes are cultured with the composition. In certain embodiments, the lymphocytes are cultured with about 10 μg/ul to about 1,000 μg/ml of the composition. In certain embodiments, the lymphocytes are cultured with the composition for about 48 hours or less.

In certain embodiments, determining the percentage of the stimulated or the unstimulated lymphocytes that are CD4+ and express: (i) CD134; (ii) CD137; or (iii) CD134 and CD137 is performed by flow cytometry.

The present disclosure provides a method for determining the propensity of a composition to elicit the production of antibodies specific to said composition relative to a reference propensity. In certain embodiments, the method can include (a) culturing antigen presenting cells (APCs) in the presence of the composition to generate stimulated APCs; (b) culturing APCs in the absence of the composition to generate unstimulated APCs; (c) separately culturing the stimulated APCs with CD4+ lymphocytes and the unstimulated APCs with CD4+ lymphocytes; (d) determining the percentage of the CD4+ lymphocytes cultured with the stimulated APCs that express: (i) CD134; (ii) CD137; or (iii) CD134 and CD137; (e) determining the percentage of the CD4+ lymphocytes cultured with the unstimulated APCs that express: (i) CD134; (ii) CD137; or (iii) CD134 and CD137; and (f) calculating a stimulation index value. In certain embodiments, when the stimulation index value in (f) is greater than or equal to a reference stimulation index value then the composition has a greater propensity to elicit antibodies specific to said composition and when the stimulation index value in (f) is less than the reference stimulation index value then the composition has a lesser propensity to elicit antibodies specific to said composition. In certain embodiments, the stimulation index value is determined by dividing the percentage of CD4+ lymphocytes determined in (d) with the percentage of CD4+ lymphocytes determined in (e). In certain embodiments, the stimulation index value is determined by outlier sum analysis or determined by linear regression. In certain embodiments, the APCs are obtained from a single donor. In certain embodiments, the APCs are obtained from about 20 donors to about 50 donors. In certain embodiments, the APCs are obtained from about 35 to about 45 donors. In certain embodiments, the APCs are obtained from at least about 20 donors, at least about 25 donors, at least about 30 donors, at least about 35 donors, at least about 40 donors or at least about 45 donors.

The present disclosure further provides a method for determining the propensity of a composition to elicit the production of antibodies specific to the composition, wherein the method comprises: (a) separately culturing APCs from individual donors in the presence of the composition to generate stimulated APCs; (b) separately culturing APCs from the individual donors in the absence of the composition to generate unstimulated APCs; (c) separately culturing the stimulated APCs with CD4+ lymphocytes and the unstimulated APCs with CD4+ lymphocytes; (d) determining the percentage of the CD4+ lymphocytes cultured with the stimulated APCs that express: (i) CD134; (ii) CD137; or (iii) CD134 and CD137; (e) determining the percentage of the CD4+ lymphocytes cultured with the unstimulated APCs that express: (i) CD134; (ii) CD137; or (iii) CD134 and CD137; (f) calculating a stimulation index value for each of the donors; and (g) calculating a number of reactive lymphocyte donors where the donors' stimulation index value is greater than or equal to a reference value stimulation index value and the number of non-reactive lymphocyte donors where the donors' stimulation index value is less than the reference stimulation index value. In certain embodiments, the composition has a high propensity to elicit the production of antibodies specific to the composition if the number of reactive donors is greater than 30% of the total number of donors and the composition has a low propensity to elicit the production of antibodies specific to the composition if the number of reactive donors is less than 20% of the total number of donors. In certain embodiments, the stimulation index value is determined by dividing the percentage of CD4+ lymphocytes of an individual donor determined in (d) with the percentage of CD4+ lymphocytes of that individual donor determined in (e). In certain embodiments, the stimulation index value is determined by outlier sum analysis or determined by linear regression. In certain embodiments, the stimulation index value is determined by dividing the percentage of CD4+ lymphocytes of an individual donor determined in (d) with the percentage of CD4+ lymphocytes of that individual donor determined in (e). In certain embodiments, the APCs are obtained from about 20 donors to about 50 donors. In certain embodiments, the APCs are obtained from about 35 to about 45 donors. In certain embodiments, the APCs are obtained from at least about 20 donors, at least about 25 donors, at least about 30 donors, at least about 35 donors, at least about 40 donors or at least about 45 donors.

The present disclosure provides a method for determining the propensity of a neoantigen to elicit an immune response specific to said neoantigen relative to a reference antigen. In certain embodiments, the method includes (a) culturing APCs in the presence of the neoantigen to generate stimulated APCs; (b) culturing APCs in the absence of the neoantigen to generate unstimulated APCs; (c) separately culturing the stimulated APCs with CD4+ lymphocytes and the unstimulated APCs with CD4+ lymphocytes; (d) determining the percentage of the CD4+ lymphocytes cultured with the stimulated APCs that express: (i) CD134; (ii) CD137; or (iii) CD134 and CD137; (e) determining the percentage of the CD4+ lymphocytes cultured with the unstimulated APCs that express: (i) CD134; (ii) CD137; or (iii) CD134 and CD137; and (f) calculating a stimulation index value. In certain embodiments, when the stimulation index value in (f) is greater than or equal to a reference stimulation index value then the neoantigen has a greater propensity to elicit an immune response specific to said neoantigen and when the stimulation index value in (f) is less than the reference stimulation index value then the neoantigen has a lesser propensity to elicit an immune response specific to said neoantigen. In certain embodiments, the neoantigen is present in a complex with an MHC class II molecule. In certain embodiments, the stimulation index value is determined by dividing the percentage of CD4+ lymphocytes determined in (d) with the percentage of CD4+ lymphocytes determined in (e). In certain embodiments, the stimulation index value is determined by outlier sum analysis or determined by linear regression. In certain embodiments, the APCs are obtained from about 20 donors to about 50 donors. In certain embodiments, the APCs are obtained from about 35 to about 45 donors. In certain embodiments, the APCs are obtained from at least about 20 donors, at least about 25 donors, at least about 30 donors, at least about 35 donors, at least about 40 donors or at least about 45 donors.

In certain embodiments, the reference stimulation index value is from about 1.0 to about 4.0, from about 1.0 to about 3.0 or from about 1.8 to about 3.0. In certain embodiments, the reference stimulation index value is about 1.6 or greater, about 1.7 or greater, about 1.8 or greater, about 1.9 or greater, about 2.0 or greater, about 2.1 or greater, about 2.2 or greater, about 2.3 or greater, about 2.4 or greater, about 2.5 or greater, about 2.6 or greater, about 2.7 or greater, about 2.8 or greater, about 2.9 or greater or about 3.0 or greater.

In certain embodiments, the CD4+ lymphocytes comprise CD8− T cells. In certain embodiments, at least 10% of the CD4+ lymphocytes are CD8− T cells.

In certain embodiments, the composition comprises a peptide, a polypeptide or a small molecule compound. In certain embodiments, the peptide or polypeptide comprises a neoantigen. In certain embodiments, the polypeptide is an antibody or fragment thereof. In certain embodiments, the antibody is a human, humanized or chimeric antibody. In certain embodiments, the composition is an antibody-drug conjugate (ADC).

In certain embodiments, about 1×10⁵ to about 1×10⁷ APCs are cultured with the composition and/or neoantigen. In certain embodiments, the APCs are cultured with about 10 μg/ul to about 1,000 μg/ml of the composition and/or neoantigen. In certain embodiments, the APCs are cultured with the composition and/or neoantigen for about 48 hours or less.

In certain embodiments, determining the percentage of the CD4+ lymphocytes that express: (i) CD134; (ii) CD137; or (iii) CD134 and CD137 is performed by flow cytometry.

The present disclosure further provides kits for performing any one of the methods of disclosed herein.

### BRIEF DESCRIPTION OF THE FIGURES

FIG. 1 displays a schematic of a non-limiting embodiment of a method for determining the propensity of a composition to elicit production of ADAs.

FIG. 2 displays the FACS analysis of two different antibodies, AVASTIN® and bococizumab.

FIG. 3 displays the analysis of six (6) antibodies, AVASTIN®, GNE-αPCSK9 (also referred to as RG7652), alirocumab (PRALUENT®), evolocumab (REPATHA®), bococizumab and HA33, with different clinical ADA rates.

FIG. 4A displays the number of donors that expressed CD134 for AVASTIN®, HA33 and KLH.

FIG. 4B displays the number of donors that expressed CD137 for AVASTIN®, HA33 and KLH.

FIG. 4C displays the number of donors that expressed CD134 and CD137 for AVASTIN®, HA33 and KLH.

FIG. 4D displays the number of donors that expressed CD134 and/or CD137 for AVASTIN®, HA33 and KLH.

FIG. 5 displays that there is a correlation between the predicted immunogenicity as determined by the presently disclosed assay and the observed immunogenicity in the clinic.

FIG. 6A displays a schematic showing the antibody blocking of HLA-DR and HLA-II.

FIG. 6B displays the number of positive donors upon blocking of HLA-DR and HLA-II.

FIG. 7 displays that there is no correlation between IL-2 secretion in vitro and clinical immunogenicity.

FIG. 8 displays that there is no correlation between cytokine secretion in vitro and clinical immunogenicity.

FIG. 9 displays a schematic of a non-limiting embodiment of methods of the present disclosure for determining the propensity of a composition to elicit production of ADAs where isolated APCs are initially cultured with the composition and then those APCs are subsequently co-cultured with T cells and activation of the T cells is used to determine the propensity of the composition to elicit production of ADAs.

FIG. 10 displays a schematic of a non-limiting rapid embodiment of the methods of the present disclosure for determining the propensity of a composition to elicit production of ADAs where the APCs are initially cultured with the composition and then subsequently co-cultured with T cells and activation of the T cells is used to determine the propensity of the composition to elicit production of ADAs.

FIG. 11A displays the analysis of four (4) bispecific antibodies that have an antigen-binding domain that is specific for T cells. The stimulation index (SI) value line indicates values greater than 1.8.

FIG. 11B displays the analysis of four (4) bispecific antibodies that have an antigen-binding domain that is specific for T cells. The SI value line indicates values greater than 3.

FIG. 12A displays the analysis of two (2) bispecific antibodies that have an antigen-binding domain that is specific for T cells. The SI value line indicates values greater than 1.8.

FIG. 12B displays the analysis of two (2) bispecific antibodies that have an antigen-binding domain that is specific for T cells. The SI value line indicates values greater than 3.

FIG. 13A displays a schematic of the T cell-activation assay that includes HLA blockage to show that the proposed immunogenicity is due to the therapeutic-specific activation of T cells.

FIG. 13B displays the analysis of the bispecific antibody TDB2 using the assay depicted in FIG. 13A.

FIG. 14 displays the analysis of a bispecific antibody produced by expression of its two antigen-binding domains in a single cell (TDB4A) or produced by a two cell system, where each cell expresses one of the two antigen-binding domains of the bispecific antibody (TDB4B).

### DETAILED DESCRIPTION

For clarity, but not by way of limitation, the detailed description of the presently disclosed subject matter is divided into the following subsections:

I. Definitions;

II. Methods;

III. Compositions;

IV. Kits; and

V. Exemplary Embodiments.

### I. Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

As used herein, the use of the word “a” or “an” when used in conjunction with the term “comprising” in the claims and/or the specification can mean “one,” but it is also consistent with the meaning of “one or more,” “at least one,” and “one or more than one.” Still further, the terms “having,” “including,” “containing” and “comprising” are interchangeable and one of skill in the art is cognizant that these terms are open ended terms.

The term “about” or “approximately,” as used herein, can mean within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. Where particular values are described in the application and claims, unless otherwise stated the term “about” can mean an acceptable error range for the particular value, such as ±10% of the value modified by the term “about.”

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g., scFv); and multispecific antibodies formed from antibody fragments.

An antibody “which binds” an antigen of interest is one that binds the antigen with sufficient affinity such that the antibody is useful as an assay reagent, e.g., as a detection antibody. Typically, such an antibody does not significantly cross-react with other polypeptides. With regard to the binding of a polypeptide to a target molecule, the term “specific binding” or “specifically binds to” or is “specific for” a particular polypeptide or an epitope on a particular polypeptide target means binding that is measurably different from a non-specific interaction. Specific binding can be measured, for example, by determining binding of a target molecule compared to binding of a control molecule, which generally is a molecule of similar structure that does not have binding activity.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{d}). Affinity can be measured by common methods known in the art, including those described herein. Specific illustrative and exemplary embodiments for measuring binding affinity are described in the following.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

The term “cytotoxic agent” as used herein refers to a substance that inhibits or prevents a cellular function and/or causes cell death or destruction. Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anticancer agents disclosed below.

A “detection antibody,” as used herein, refers to an antibody that specifically binds a target molecule in a sample. Under certain conditions, the detection antibody forms a complex with the target molecule. A detection antibody is capable of being detected either directly through a label, which may be detected, or indirectly, e.g., through use of another antibody that is labeled and that binds the detection antibody. For direct labeling, the detection antibody is typically conjugated to a moiety that is detectable by some means, for example, including but not limited to, fluorophore.

The term “detecting,” is used herein, to include both qualitative and quantitative measurements of a target molecule or processed forms thereof. In certain embodiments, detecting includes identifying the mere presence of the target molecule as well as determining whether the target molecule is present at detectable levels.

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g., B cell receptor); and B cell activation.

The term “Fc region” herein is used to define a C-terminal region of an immunoglobulin heavy chain that contains at least a portion of the constant region. The term includes native sequence Fc regions and variant Fc regions. In certain embodiments, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

“Framework” or “FR” refers to variable domain residues other than hypervariable region (CDR) residues. The FR of a variable domain generally consists of four FR domains: FR1, FR2, FR3, and FR4. Accordingly, the CDR and FR sequences generally appear in the following sequence in VH (or VL): FR1-H1(L1)-FR2-H2(L2)-FR3-H3(L3)-FR4.

The terms “full-length antibody,” “intact antibody” and “whole antibody” are used herein interchangeably to refer to an antibody having a structure substantially similar to a native antibody structure or having heavy chains that contain an Fc region as defined herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda Md. (1991), Vols. 1-3. In certain embodiments, for the VL, the subgroup is subgroup kappa I as in Kabat et al., supra. In certain embodiments, for the VH, the subgroup is subgroup III as in Kabat et al., supra.

A “humanized” antibody refers to a chimeric antibody comprising amino acid residues from non-human CDRs and amino acid residues from human FRs. In certain embodiments, a humanized antibody will comprise substantially all of at least one, and typically two, variable domains, in which all or substantially all of the CDRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “CDR,” as used herein, refers to each of the regions of an antibody variable domain which are hypervariable in sequence (also referred to herein as “complementarity determining regions” or “CDRs”) and/or form structurally defined loops (“hypervariable loops”) and/or contain the antigen-contacting residues (“antigen contacts”). Unless otherwise indicated, CDR residues and other residues in the variable domain (e.g., FR residues) are numbered herein according to Kabat et al., supra. Generally, antibodies comprise six CDRs: three in the VH (H1, H2, H3), and three in the VL (L1, L2, L3). Exemplary CDRs herein include:

(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));

(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, Md. (1991));

(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)); and

(d) combinations of (a), (b), and/or (c), including CDR amino acid residues 46-56 (L2), 47-56 (L2), 48-56 (L2), 49-56 (L2), 26-35 (H1), 26-35b (H1), 49-65 (H2), 93-102 (H3), and 94-102 (H3).

An “immunoconjugate” refers to an antibody conjugated to one or more heterologous molecule(s), including but not limited to a cytotoxic agent.

An “individual,” “subject” or “donor” herein is a vertebrate, such as a human or non-human animal, for example, a mammal. Mammals include, but are not limited to, humans, non-human primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys. In certain embodiments, the individual, subject or donor is a human.

As used herein, the term “in vitro” refers to an artificial environment and to processes or reactions that occur within an artificial environment. In vitro environments exemplified, but are not limited to, test tubes and cell cultures.

As used herein, the term “in vivo” refers to the natural environment (e.g., an animal or a cell) and to processes or reactions that occur within a natural environment, such as embryonic development, cell differentiation, neural tube formation, etc.

An “isolated” antibody is one which has been separated from a component of its natural environment. In certain embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., I Chromatogr. B 848:79-87 (2007).

The terms “label” or “detectable label,” as used herein, refers to any chemical group or moiety that can be linked to a substance that is to be detected or quantitated, e.g., an antibody. A label is a detectable label that is suitable for the sensitive detection or quantification of a substance. Non-limiting examples of detectable labels include, but are not limited to, luminescent labels, e.g., fluorescent, phosphorescent, chemiluminescent, bioluminescent and electrochemiluminescent labels, radioactive labels, enzymes, particles, magnetic substances, electroactive species and the like. Alternatively, a detectable label can signal its presence by participating in specific binding reactions. Non-limiting examples of such labels include haptens, antibodies, biotin, streptavidin, his-tag, nitrilotriacetic acid, glutathione S-transferase, glutathione and the like.

The term “monoclonal antibody,” as used herein, refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the presently disclosed subject matter may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

The term “nucleic acid molecule” or “polynucleotide” includes any compound and/or substance that comprises a polymer of nucleotides. Each nucleotide is composed of a base, specifically a purine- or pyrimidine base (i.e., cytosine (C), guanine (G), adenine (A), thymine (T) or uracil (U)), a sugar (i. e., deoxyribose or ribose), and a phosphate group. Often, the nucleic acid molecule is described by the sequence of bases, whereby said bases represent the primary structure (linear structure) of a nucleic acid molecule. The sequence of bases is typically represented from 5′ to 3′. Herein, the term nucleic acid molecule encompasses deoxyribonucleic acid (DNA) including, e.g., complementary DNA (cDNA) and genomic DNA, ribonucleic acid (RNA), in particular messenger RNA (mRNA), synthetic forms of DNA or RNA, and mixed polymers comprising two or more of these molecules. The nucleic acid molecule can be linear or circular. In addition, the term nucleic acid molecule includes both, sense and antisense strands, as well as single stranded and double stranded forms. Moreover, the herein described nucleic acid molecule can contain naturally occurring or non-naturally occurring nucleotides. Examples of non-naturally occurring nucleotides include modified nucleotide bases with derivatized sugars or phosphate backbone linkages or chemically modified residues. Nucleic acid molecules also encompass DNA and RNA molecules which are suitable as a vector for direct expression of an antibody of the present disclosure in vitro and/or in vivo, e.g., in a host or patient. Such DNA (e.g., cDNA) or RNA (e.g., mRNA) vectors, can be unmodified or modified. For example, mRNA can be chemically modified to enhance the stability of the RNA vector and/or expression of the encoded molecule so that mRNA can be injected into a subject to generate the antibody in vivo (see, e.g., Stadler et al., Nature Medicine 2017, published online 12 Jun. 2017, doi:10.1038/nm.4356 or EP 2 101 823 B1).

“Purified” polypeptide (e.g., antibody), as used herein, refers to a polypeptide that has been increased in purity, such that it exists in a form that is more pure than it exists in its natural environment and/or when initially synthesized and/or amplified under laboratory conditions. Purity is a relative term and does not necessarily mean absolute purity.

The term “package insert,” as used herein, refers to instructions customarily included in commercial packages that contain information concerning the use of the components of the package.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The terms “polypeptide” and “protein,” as used interchangeably herein, refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation or any other manipulation or modification, such as conjugation with a labeling component. Also included within the definition are, for example, polypeptides containing one or more analogs of an amino acid (including, for example, unnatural amino acids, etc.), as well as other modifications known in the art. The terms “polypeptide” and “protein” as used herein specifically encompass antibodies.

As used herein, the term “recombinant protein” refers generally to peptides and proteins that have been genetically manipulated. In certain embodiments, such recombinant proteins are “heterologous,” i.e., foreign to the cell being utilized.

A “sample,” as used herein, refers to a small portion of a larger quantity of material. In certain embodiments, a sample includes, but is not limited to, cells in culture, cell supernatants, cell lysates, serum, blood plasma, biological fluid (e.g., blood, plasma, serum, stool, urine, lymphatic fluid, ascites, ductal lavage, saliva and cerebrospinal fluid) and tissue samples. The source of the sample may be solid tissue (e.g., from a fresh, frozen, and/or preserved organ, tissue sample, biopsy or aspirate), blood or any blood constituents, bodily fluids (such as, e.g., urine, lymph, cerebral spinal fluid, amniotic fluid, peritoneal fluid or interstitial fluid), or cells from the individual, including circulating cells.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (CDRs). (See, e.g., Kindt et al. Kuby Immunology, 6^{th} ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

### II. Methods

The presently disclosed subject matter provides methods for determining the propensity of a composition that comprises a therapeutic, e.g., a polypeptide or a fragment thereof, to elicit production of anti-drug antibodies (ADAs). In certain embodiments, the presently disclosed methods can be used to determine the propensity of a composition comprising an antibody or fragment thereof or an antibody-drug conjugate (ADC) to elicit production of ADAs. The present disclosure also provides kits for performing the methods disclosed herein.

In certain embodiments, the methods of the present disclosure can be used to identify polypeptide variants, e.g., antibody variants, that have a reduced propensity to elicit production of ADAs compared to the parent polypeptide, e.g., a parent antibody. In certain embodiments, the methods disclosed herein can be used to analyze newly-developed polypeptides, e.g., antibodies. For example, but not by way of limitation, the methods disclosed herein can be used to identify a polypeptide, e.g., an antibody, that has a lower propensity to elicit ADA from a larger repertoire of polypeptides that specifically bind to the same antigen. In certain embodiments, methods of the present disclosure can be used to determine the immunogenic potential of a newly developed polypeptide, e.g., antibody, prior to clinical studies. In certain embodiments, the present disclosed methods can be used to determine the immunogenicity potential of aggregates of a polypeptide, e.g., antibody. In certain embodiments, the presently disclosed methods can be used to analyze the immunogenicity of sequence variants of an antibody, e.g., those that arise during the manufacture and/or production of the antibody. In certain embodiments, the presently disclosed methods can be used to analyze the immunogenicity of a neoantigen. In certain embodiments, the presently disclosed methods can be used to analyze the immunogenicity of a peptide.

In certain embodiments, methods of the present disclosure can include culturing lymphocytes in the presence of the composition to generate stimulated lymphocytes. In certain embodiments, the method can further include culturing lymphocytes in the absence of the composition to generate unstimulated lymphocytes. For example, but not by way of limitation, the lymphocytes can be cultured in the presence of the composition, e.g., for about 24 to about 72 hours. In certain embodiments, the lymphocytes can be cultured in the presence of the polypeptide from about 12 to about 72 hours, about 12 to about 60 hours, about 12 hours to about 48 hours, about 12 hours to about 24 hours, about 24 hours to about 72 hours, about 24 hours to about 60 hours, about 24 to about 48 hours, about 48 hours to about 72 hours or about 48 hours to about 60 hours. In certain embodiments, the lymphocytes can be cultured in the presence of the composition for about 48 hours or less.

The concentration of lymphocytes used in the presently disclosed methods can depend on the size of the culture dishes and/or plates used. For example, but not by way of limitation, lymphocytes can be used at a concentration from about 1×10⁵ to about 1×10⁷ cells/ml, e.g., about 2×10⁶ cells/ml, e.g., in a 24-well plate and/or 96-well plate. In certain embodiments, the number of lymphocytes used can be from about 1×10⁵ to about 9×10⁶ cells, from about 3×10⁵ to about 8×10⁶ cells, from about 3×10⁵ to about 7×10⁶ cells, from about 4×10⁵ to about 6×10⁶ cells, from about 5×10⁵ to about 5×10⁶ cells, from about 6×10⁵ to about 4×10⁶ cells, from about 7×10⁵ to about 3×10⁶ cells, from about 8×10⁵ to about 2×10⁶ cells, from about 9×10⁵ to about 2×10⁶ cells or from about 9×10⁵ to about 1×10⁶ cells. In certain embodiments, about 1×10⁶ cells lymphocytes are used. In certain embodiments, the number of lymphocytes used can be from about 1×10⁵ cells to about 3×10⁵ cells, e.g., about 2×10⁵ cells lymphocytes are used. In certain embodiments, lymphocytes can be used at a concentration from about 0.1×10⁶ cells/ml to about 1×10⁶ cells/ml, e.g., about 0.2×10⁶ cells/ml to about 0.4×10⁶ cells/ml.

Lymphocytes for use in the presently disclosed methods include any cell that can interact with an antigen complexed with a major histocompatibility complex (MHC) on the surface of a cell. For example, but not by way of limitation, the lymphocytes can comprise T cells. For example, but not by way of limitation, at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98% or about 99% of the lymphocytes are T cells. In certain embodiments, at least about 20%, e.g., at least about 30%, of the lymphocytes are T cells, e.g., CD4+ T cells. In certain embodiments, the lymphocytes can further include antigen-presenting cells (APCs).

In certain embodiments, the T cells are CD8−. In certain embodiments, the T cells are CD4+. In certain embodiments, the T cells are CD4+CD8−. For example, but not by way of limitation, at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98% or about 99% of the T cells are CD8−, CD4+ or CD4+CD8− cells. In certain embodiments, at least about 20%, e.g., at least about 30%, of the T cells are CD8−, CD4+ or CD4+CD8− cells.

Non-limiting sources of lymphocytes include peripheral blood mononuclear cells (PBMCs) isolated from donors. In certain embodiments, the PBMCs are isolated from a sample of a donor, e.g., from a blood sample of the donor. PBMCs can be isolated from a sample of a donor by any method known in the art, e.g., by a density gradient centrifugation. In certain embodiments, PBMCs are initially isolated from a sample of a donor and then subjected to a CD8 negative selection to isolate and/or select CD8− cells, e.g., CD8− T cells. In certain embodiments, PBMCs can be a PBMC cell line. In certain embodiments, the lymphocytes can comprise T cells, e.g., CD8− T cells, CD4+ T cells or CD4+CD8− T cells. In certain embodiments, the lymphocytes can be differentiated from stem cells or iPSC cells. For example, but not by way of limitation, the lymphocytes can be T cells, e.g., CD8− T cells, CD4+ T cells or CD4+CD8− T cells, that are differentiated from stem cells or iPSC cells. In certain embodiments, APCs including, but not limited to, dendritic cells, macrophages and B cells, can be isolated from PBMCs, and used in methods that include culturing the APCs in the presence of a composition of interest and/or T cells, as discussed below. Alternatively and/or additionally, the lymphocytes obtained from PBMCs can include T cells, e.g., CD8− T cells, CD4+ T cells or CD4+CD8− T cells, and APCs for use in the methods disclosed herein.

In certain embodiments, the methods of the present disclosure can comprise isolating CD14+ cells from PBMCs prior to exposure to the composition. For example, but not by way of limitation, CD14+ cells can be isolated and induced, e.g., by exposure to GM-CSF and/or IL4, to differentiate into APCs, e.g., dendritic cells, e.g., immature dendritic cells, and subsequently cultured in the presence of the composition to generate stimulated APCs, e.g., stimulated mature dendritic cells. In certain embodiments, the APCs, e.g., immature dendritic cells, can be cultured in the presence of the composition and GM-CSF, IL4, TNF-α, IL-1(3, IL6 and/or PGE2 to generate stimulated APCs, e.g., stimulated mature dendritic cells. The stimulated APCs, e.g., monocyte-derived dendritic cells, can then be cultured with T cells, e.g., CD4+ T cells and/or CD4+CD8− T cells, and T cell activation can be assayed. In certain embodiments, the T cells can be isolated from PBMCs, as discussed below. In certain embodiments, APCs and T cells can be isolated from the same PBMC sample or population. In certain embodiments, APCs and T cells can be isolated from the same donor. In certain embodiments, culturing of the APCs with the composition in the absence of T cells followed by the subsequent culturing of APCs with T cells can avoid potential assay interference due to the direct activation of the T cell by the composition.

In certain embodiments, the lymphocytes are cultured with about 10 μg/ul to about 1,000 μg/ml of the composition, e.g., about 100 μg/ml of the composition. For example, but not by way of limitation, the composition can be used at a concentration from about 30 μg/ml to about 1,000 μg/ml, from about 40 μg/ml to about 1,000 μg/ml, from about 50 μg/ml to about 1,000 μg/ml, from about 60 μg/ml to about 1,000 μg/ml, from about 70 μg/ml to about 1,000 μg/ml, from about 80 μg/ml to about 1,000 μg/ml, from about 90 μg/ml to about 1,000 μg/ml, from about 10 μg/ml to about 900 μg/ml, from about 10 μg/ml to about 800 μg/ml, from about 10 μg/ml to about 700 μg/ml, from about 10 μg/ml to about 600 μg/ml, from about 10 μg/ml to about 500 μg/ml, from about 10 μg/ml to about 400 μg/ml, from about 10 μg/ml to about 300 μg/ml, from about 10 μg/ml to about 200 μg/ml, from about 50 μg/ml to about 150 μg/ml or from about 75 μg/ml to about 125 μg/ml. In certain embodiments, the lymphocytes are cultured with about 100 μg/ml of the composition. In certain embodiments, the lymphocytes can be T cells, e.g., CD4+ T cells and/or CD4+CD8− T cells, which are cultured with the composition. In certain embodiments, the lymphocytes can include T cells and APCs, which are cultured with the composition. Alternatively and/or additionally, the method can include culturing APCs, e.g., dendritic cells, macrophages and/or B cells, in the presence of the composition of interest and the lymphocytes, e.g., T cells. In certain embodiments, the method can include culturing dendritic cells in the presence of the composition of interest and the lymphocytes, e.g., T cells. In certain embodiments, the methods can include culturing isolated APCs, e.g., dendritic cells, macrophages and/or B cells, in the presence of the composition of interest but in the absence of T cells. In certain embodiments, the methods can include culturing isolated dendritic cells in the presence of the composition of interest but in the absence of T cells.

In certain embodiments, the method can further include determining the percentage of the stimulated lymphocytes that are CD4+ and express: (i) CD134; (ii) CD137; or (iii) CD134 and CD137. In certain embodiments, the method can include determining the percentage of the unstimulated lymphocytes that are CD4+ and express: (i) CD134; (ii) CD137; or (iii) CD134 and CD137. In certain embodiments, the method can include determining whether such cells are live. In certain embodiments, determining the amount of unstimulated and/or stimulated lymphocytes includes (i) contacting the lymphocytes, e.g., T cells, with one or more detection agent that bind to the CD4, CD134 and/or CD137 and (ii) determining the number of lymphocytes, e.g., T cells, that are bound to the one or more detection agents.

In certain embodiments, the detection agent for use in the methods disclosed herein is an antibody (also referred to herein as “a detection antibody”). In certain embodiments, the detection agent specifically binds to the polypeptide analyzed by the disclosed methods, e.g., the detection agent specifically binds to an epitope present on the polypeptide or fragment thereof. In certain embodiments, the detection agent is an antibody that binds to CD4. In certain embodiments, the detection agent is an antibody that binds to CD134. In certain embodiments, the detection agent is an antibody that binds to CD137. In certain embodiments, the detection agents used in the assay methods disclosed herein can be used at a concentration from about 0.05 μg/ml to about 5 μg/ml, e.g., about 1 μg/ml.

In certain embodiments, the detection agents, e.g., detection antibodies, for use in the disclosed methods can be labeled. Labels include, but are not limited to, labels or moieties that are detected directly, such as fluorescent, chromophoric, electron-dense, chemiluminescent, and radioactive labels, as well as moieties, such as enzymes or ligands, that are detected indirectly, e.g., through an enzymatic reaction or molecular interaction. Non-limiting examples of labels include the radioisotopes ³²P, ¹⁴C, ¹²⁵I, ³H and ¹³¹I, fluorophores such as rare earth chelates or fluorescein and its derivatives, rhodamine and its derivatives, dansyl, umbelliferone, luciferases, e.g., firefly luciferase and bacterial luciferase (see U.S. Pat. No. 4,737,456), luciferin, 2,3-dihydrophthalazinediones, horseradish peroxidase (HRP), alkaline phosphatase, β-galactosidase, glucoamylase, lysozyme, saccharide oxidases, e.g., glucose oxidase, galactose oxidase, and glucose phosphate dehydrogenase, heterocyclic oxidases such as uricase and xanthine oxidase, coupled with an enzyme that employs hydrogen peroxide to oxidize a dye precursor such as HRP, lactoperoxidase or microperoxidase, biotin/avidin, spin labels, bacteriophage labels, stable free radicals and the like. In certain embodiments, the detection agent, e.g., antibody, is labeled with a fluorophore.

In certain embodiments, determining the number of cells, e.g., lymphocytes, labeled by the one or more detection agents is performed by any method that is able to detect the detection agent. In certain embodiments, the detection agent can be detected by monitoring the label of the detection agent, e.g., the fluorescent label. In certain embodiments, determining the number of cells lymphocytes labeled by the one or more detection agents is performed by flow cytometry.

In certain embodiments, the method further includes calculating a stimulation index value. In certain embodiments, the stimulation index value can be determined by dividing the percentage of stimulated lymphocytes with the percentage of unstimulated lymphocytes. Alternatively or additionally, the stimulation index value can be determined by outlier sum analysis or by linear regression. In certain embodiments, the stimulation index value can be determined by dividing the maximum value or average value of stimulated lymphocytes with the maximum value or average value of unstimulated lymphocytes.

In certain embodiments, the method can include comparing the stimulation index value to a reference stimulation index. In certain embodiments, when the stimulation index value is greater than the reference stimulation index value, the polypeptide has a greater propensity to elicit production of ADAs than the reference. Alternatively, when the stimulation index value of a polypeptide is less than the reference stimulation index value, the polypeptide has a lesser propensity to elicit production of ADAs than the reference, e.g., in the clinical setting.

In certain embodiments, the reference stimulation index is indicative of a known propensity to elicit the production of ADAs, e.g., in the clinical setting. In certain embodiments, the reference stimulation index is from about 1.0 to about 4.0, e.g., from about 1.0 to about 3.0, from about 1.1 to about 2.0, from about 1.2 to about 2.0, from about 1.3 to about 2.0, from about 1.4 to about 2.0, from about 1.5 to about 2.0, from about 1.6 to about 2.0, from about 1.7 to about 2.0, from about 1.8 to about 2.0 or from about 1.8 to about 3.0. In certain embodiments, the reference stimulation index is from about 1.5 to about 2.0. In certain embodiments, the reference stimulation index is from about 1.6 to about 1.8. In certain embodiments, the reference stimulation index value is about 1.6 or greater. In certain embodiments, the reference stimulation index value is about 1.7 or greater. In certain embodiments, the reference stimulation index value is about 1.8 or greater. In certain embodiments, the reference stimulation index value is about 1.9 or greater. In certain embodiments, the reference stimulation index value is about 2.0 or greater. In certain embodiments, the reference stimulation index value is about 2.1 or greater. In certain embodiments, the reference stimulation index value is about 2.2 or greater. In certain embodiments, the reference stimulation index value is about 2.3 or greater. In certain embodiments, the reference stimulation index value is about 2.4 or greater. In certain embodiments, the reference stimulation index value is about 2.5 or greater. In certain embodiments, the reference stimulation index value is about 2.6 or greater. In certain embodiments, the reference stimulation index value is about 2.7 or greater. In certain embodiments, the reference stimulation index value is about 2.8 or greater. In certain embodiments, the reference stimulation index value is about 2.9 or greater. In certain embodiments, the reference stimulation index value is about 3.0 or greater.

In certain embodiments, the reference stimulation index is a value produced by a composition (e.g., a reference composition) that has a low propensity to elicit production of ADAs, e.g., in the clinical setting. For example, but not by way of limitation, the reference composition can be an antibody that has been shown to not elicit production of ADAs in a clinical setting or has a low propensity to elicit production of ADAs in a clinical setting. Alternatively or additionally, the reference stimulation index can be the stimulation index of a composition (e.g., reference composition) that has been shown to elicit production of ADAs. For example, but not by way of limitation, the reference composition can be an antibody that has been shown to elicit production of ADAs in a clinical setting. In certain embodiments, the reference composition can be an antibody disclosed in any one of the Figures and/or examples. Alternatively or additionally, in the context of antibody variants, the reference stimulation index can be the stimulation index of the parent antibody. In certain embodiments, in the context of bispecific antibodies, the reference stimulation index can be the stimulation index of one of the parent antibodies.

In certain embodiments, methods of the present disclosure can include (i) culturing lymphocytes in the presence of the composition to generate stimulated lymphocytes; (ii) culturing lymphocytes in the absence of the composition to generate unstimulated lymphocytes; (iii) determining the percentage of the stimulated lymphocytes that are CD4+ and express: (a) CD134; (b) CD137; or (c) CD134 and CD137; (iv) determining the percentage of the unstimulated lymphocytes that are CD4+ and express: (a) CD134; (b) CD137; or (c) CD134 and CD137; and (v) calculating a stimulation index value. In certain embodiments, when the stimulation index value in (v) is greater than or equal to a reference stimulation index value then the composition has a greater propensity to elicit antibodies specific to said composition. In certain embodiments, when the stimulation index value in (v) is less than the reference stimulation index value then the composition has a lesser propensity to elicit antibodies specific to said composition.

In certain embodiments, methods of the present disclosure can include culturing APCs, e.g., CD14+ APCs, in the presence of the composition and in the absence of CD4+ lymphocytes, e.g., T cells. In certain embodiments, the APCs are isolated from PBMCs prior to the culturing of such APCs with the composition. In certain embodiments, the methods can also include culturing APCs, e.g., isolated APCs, in the absence of the composition and in the absence of CD4+ lymphocytes, e.g., T cells. In certain embodiments, the method can further include co-culturing CD4+ lymphocytes, e.g., T cells, with the APCs that have been previously cultured in the presence of the composition to produce stimulated CD4+ lymphocytes, e.g., stimulated T cells. In certain embodiments, the method can further include co-culturing CD4+ lymphocytes, e.g., T cells, with the APCs that have been previously cultured in the absence of the composition to produce unstimulated CD4+ lymphocytes, e.g., unstimulated T cells. In certain embodiments, the CD4+ lymphocytes, e.g., CD4+ T cells and/or CD4+CD8− T cells, are isolated from PBMCs. In certain embodiments, the T cells and the APCs are isolated from the same PBMC population. In certain embodiments, the T cells, e.g., CD4+ T cells, and the APCs, e.g., CD14+ APCs, are autologous. In certain embodiments, the APCs are dendritic cells.

In certain embodiments, a method of the present disclosure can include (i) culturing APCs in the presence of the composition to produce APCs that display an antigen of the composition; (ii) culturing APCs in the absence of the composition to produce APCs that do not present an antigen of the composition; (iii) co-culturing the APCs of (i) with CD4+ lymphocytes; (iv) co-culturing the APCs of (ii) with CD4+ lymphocytes; (v) determining the percentage of the CD4+ lymphocytes from the co-culture of (iii) that are CD4+ and express: (a) CD134; (b) CD137; or (c) CD134 and CD137; and (vi) determining the percentage of the T cells from the co-culture of (iv) that are CD4+ and express: (a) CD134; (b) CD137; or (c) CD134 and CD137; and (vii) calculating a stimulation index value. In certain embodiments, the method can further include comparing the stimulation index value of (vii) to a reference stimulation index value. In certain embodiments, when the stimulation index value in (vii) is greater than or equal to the reference stimulation index value then the composition has a greater propensity to elicit antibodies specific to said composition. In certain embodiments, when the stimulation index value in (vii) is less than the reference stimulation index value then the composition has a lesser propensity to elicit antibodies specific to said composition.

In certain embodiments, the method can include analyzing the composition with lymphocytes obtained from more than one donor. In certain embodiments, a method of the present disclosure can include analyzing the propensity of a composition to elicit production of ADAs by (i) separately culturing lymphocytes derived from individual donors with the composition of interest to generate stimulated lymphocytes and (ii) separately culturing lymphocytes derived from individual donors in the absence of the composition to generate unstimulated lymphocytes.

For example, but not by way of limitation, the lymphocytes (e.g., PBMCs, APCs, and/or T cells) used in connection with the methods of the present disclosure can be derived from at least 2 or more, at least 3 or more, at least 4 or more, at least 5 or more, at least 6 or more, at least 7 or more, at least 8 or more, at least 9 or more, at least 10 or more, at least 15 or more, at least 20 or more, at least 25 or more, at least 30 or more, at least 35 or more, at least 40 or more or at least 45 or more individual donors. In certain embodiments, such lymphocytes, e.g., PBMCs or APCs, can be separately cultured with the composition of interest. In certain embodiments, lymphocytes derived from about 20 to about 50 donors can be used, e.g., separately cultured with the composition of interest. In certain embodiments, lymphocytes derived from about 35 to about 45 donors can be separately used, e.g., cultured with the composition of interest. In certain embodiments, the lymphocytes derived from individual donors can be T cells, e.g., CD4+ T cells and/or CD4+CD8− T cells, which are cultured with the composition. In certain embodiments, the lymphocytes derived from individual donors can include T cells and APCs, which are cultured with the composition. Alternatively and/or additionally, the method can include culturing APCs, e.g., dendritic cells, macrophages and/or B cells, in the presence of the composition of interest and the lymphocytes derived from individual donors, e.g., T cells.

In certain embodiments, the method can further include (a) determining the percentage of the stimulated lymphocytes from the individual donors that are CD4+ and express: (i) CD134; (ii) CD137; or (iii) CD134 and CD137, and (b) determining the percentage of the unstimulated lymphocytes from the donors that are CD4+ and express: (i) CD134; (ii) CD137; or (iii) CD134 and CD137.

In certain embodiments, the method can further include (a) determining the percentage of the CD4+ lymphocytes cultured with the stimulated APCs that express: (i) CD134; (ii) CD137; or (iii) CD134 and CD137, and (b) determining the percentage of the CD4+ lymphocytes cultured with the unstimulated APCs that express: (i) CD134; (ii) CD137; or (iii) CD134 and CD137.

In certain embodiments, stimulation index values for each of the individual donors can be determined, e.g., by dividing the percentage of stimulated lymphocytes of an individual donor with the percentage of unstimulated lymphocytes of that individual donor.

In certain embodiments, stimulation index values for each of the individual donors can be determined, e.g., by dividing the percentage of CD4+ cells cultured with the stimulated APCs that express: (i) CD134; (ii) CD137; or (iii) CD134 and CD137 of an individual donor with the percentage of CD4+ lymphocytes cultured with the unstimulated APCs that express: (i) CD134; (ii) CD137; or (iii) CD134 and CD137 of that individual donor.

In certain embodiments, the method also includes calculating a number of reactive lymphocyte donors where the donors' stimulation index value is greater than or equal to a reference value stimulation index value and the number of non-reactive lymphocyte donors where the donors' stimulation index value is less than the reference stimulation index value. In certain embodiments, the composition has a high propensity to elicit the production of antibodies if the number of reactive donors is greater than 30% of the total number of donors. Alternatively, the composition has a low propensity to elicit the production of antibodies if the number of reactive donors is less than 20% of the total number of donors.

The present disclosure further provides methods for determining the propensity of a neoantigen to elicit an immune response to the neoantigen. In certain embodiments, the method includes (a) culturing lymphocytes in the presence of the neoantigen to generate stimulated lymphocytes; (b) culturing lymphocytes in the absence of the neoantigen to generate unstimulated lymphocytes; (c) determining the percentage of the stimulated lymphocytes that are CD4+ and express: (i) CD134; (ii) CD137; or (iii) CD134 and CD137; (d) determining the percentage of the unstimulated lymphocytes that are CD4+ and express: (i) CD134; (ii) CD137; or (iii) CD134 and CD137; and (e) calculating a stimulation index value (e.g., by dividing the percentage of stimulated lymphocytes determined in (c) with the percentage of unstimulated lymphocytes determined in (d)). In certain embodiments, when the stimulation index value in (e) is greater than or equal to a reference stimulation index value then the neoantigen has a greater propensity to elicit an immune response specific to said neoantigen and when the stimulation index value in (e) is less than the reference stimulation index value then the neoantigen has a lesser propensity to elicit an immune response specific to said neoantigen. In certain embodiments, the neoantigen is in a complex with an MHC molecule, e.g., MHC class II molecule. For example, but not by way of limitation, the neoantigen can be complexed with an MHC class II molecule

The present disclosure further provides methods for determining the propensity of a neoantigen to elicit an immune response to the neoantigen. In certain embodiments, the method includes (a) culturing APCs in the presence of the neoantigen to generate stimulated APCs; (b) culturing APCs in the absence of the neoantigen to generate unstimulated APCs; (c) separately culturing the stimulated APCs with CD4+ lymphocytes and the unstimulated APCs with CD4+ lymphocytes; (d) determining the percentage of the CD4+ lymphocytes cultured with the stimulated APCs that express: (i) CD134; (ii) CD137; or (iii) CD134 and CD137; (e) determining the percentage of the CD4+ lymphocytes cultured with the unstimulated APCs that express: (i) CD134; (ii) CD137; or (iii) CD134 and CD137; and (f) calculating a stimulation index value (e.g., by dividing the percentage of stimulated lymphocytes determined in (d) with the percentage of unstimulated lymphocytes determined in (e)). In certain embodiments, when the stimulation index value in (0 is greater than or equal to a reference stimulation index value then the neoantigen has a greater propensity to elicit an immune response specific to said neoantigen and when the stimulation index value in (0 is less than the reference stimulation index value then the neoantigen has a lesser propensity to elicit an immune response specific to said neoantigen. In certain embodiments, the neoantigen is in a complex with an MHC molecule, e.g., MHC class II molecule. For example, but not by way of limitation, the neoantigen can be complexed with an MHC class II molecule.

### III. Compositions

The present disclosure provides methods for determining the propensity of a composition to elicit production of ADAs. Non-limiting examples of such compositions that can be analyzed by the disclosed methods are provided below. For example, but not by way of limitation, the composition that is assayed using any of the methods disclosed herein can comprise a polypeptide or a fragment of the polypeptide, e.g., a peptide. In certain embodiments, the composition can comprise an antibody or a fragment thereof, e.g., a human, humanized or chimeric antibody. In certain embodiments, the composition can comprise an antibody-drug conjugate (ADC). In certain embodiments, the antibody can be a single domain antibody. In certain embodiments, the composition can comprise a neoantigen or a complex containing a neoantigen. In certain embodiments, the composition is an antibody that is specific for a neoantigen.

1. Polypeptides and Peptides

In certain embodiments, a composition analyzed by the methods disclosed herein can comprise a peptide or protein or fragment thereof.

In certain embodiments, the composition can be a protein or a fragment thereof. In certain embodiments, the protein can have a molecular weight of at least about 15-100 kD, e.g., closer to about 15 kD. In certain embodiments, the protein can include at least about 50, about 60, about 70, about 80, about 90, about 100, about 200, about 300, about 400, about 500 amino acids, about 1,000 amino acids, about 1,500 amino acids, about 2,000 amino acids, about 2,500 amino acids, about 3,000 amino acids, about 35,000 amino acids or about 40,000 amino acids. Non-limiting examples of proteins include all proteins, and, in general proteins that contain one or more disulfide bonds, including multi-chain polypeptides comprising one or more inter- and/or intrachain disulfide bonds. In certain embodiments, the proteins, e.g., antibodies, can include other post-translation modifications including, but not limited to, glycosylation and lipidation. See, e.g., Prabakaran et al., WIREs Syst Biol Med (2012), which is incorporated herein by reference in its entirety.

In certain embodiments, the composition can be a peptide. In certain embodiments, the peptides can be composed of about 3-50 amino acid residues. In certain embodiments, the 3-50 amino acid residues can be continuous within a larger polypeptide or protein or can be a group of 3-50 residues that are discontinuous in a primary sequence of a larger polypeptide or protein but that are spatially near in three-dimensional space. In certain embodiments, the peptide can be a part of a peptide, a part of a full protein or polypeptide and can be released from that protein or polypeptide by proteolytic treatment or can remain part of the protein or polypeptide.

In certain embodiments, the peptide can have a length of 3 residues or more, a length of 4 residues or more, a length of 5 residues or more, 6 residues or more, 7, residues or more, 8 residues or more, 9 residues or more, 10 residues or more, 11 residues or more, 12 residues or more, 13 residues or more, 14 residues or more, 15 residues or more, 16 residues or more, 17 residues or more, 18 residues or more, 19 residues or more, 20 residues or more, 21 residues or more, 22 residues or more, 23 residues or more, 24 residues or more, 25 residues or more, 26 residues or more, 27 residues or more, 28 residues or more, 29 residues or more, 30 residues or more, 31 residues or more, 32 residues or more, 33 residues or more, 34 residues or more, 35 residues or more, 36 residues or more, 37 residues or more, 38 residues or more, 39 residues or more, 40 residues or more, 41 residues or more, 42 residues or more, 43 residues or more, 44 residues or more, 45 residues or more, 46 residues or more, 47 residues or more, 48 residues or more, 49 residues or more or 50 residues or more. In certain embodiments, the peptide has a length of 3-50 residues, 5-50 residues, 3-45 residues, 5-45 residues, 3-40 residues, 5-40 residues, 3-35 residues, 5-35 residues, 3-30 residues, 5-30 residues, 3-25 residues, 5-25 residues, 3-20 residues, 5-20 residues, 3-15 residues, 5-15 residues, 3-10 residues, 3-10 residues, 5-10 residues, 10-15 residues, 15-20 residues, 20-25 residues, 25-30 residues, 30-35 residues, 35-40 residues, 40-45 residues or 45-50 residues. In certain embodiments, the peptide has a length of about 5 to about 30 residues.

In certain embodiments, the peptide has a length of 9 residues. In certain embodiments, the peptide has a length of 10 residues. In certain embodiments, the peptide has a length of 11 residues. In certain embodiments, the peptide has a length of 12 residues. In certain embodiments, the peptide has a length of 13 residues. In certain embodiments, the peptide has a length of 14 residues. In certain embodiments, the peptide has a length of 15 residues. In certain embodiments, the peptide has a length of 16 residues. In certain embodiments, the peptide has a length of 17 residues. In certain embodiments, the peptide has a length of 18 residues. In certain embodiments, the peptide has a length of 99 residues. In certain embodiments, the peptide has a length of 20 residues. In certain embodiments, the peptide has a length of 21 residues. In certain embodiments, the peptide has a length of 22 residues. In certain embodiments, the peptide has a length of 23 residues. In certain embodiments, the peptide has a length of 24 residues. In certain embodiments, the peptide has a length of 25 residues. In certain embodiments, the peptide has a length of 26 residues. In certain embodiments, the peptide has a length of 27 residues. In certain embodiments, the peptide has a length of 28 residues. In certain embodiments, the peptide has a length of 29 residues. In certain embodiments, the peptide, e.g., peptide, has a length of 30 residues. In certain embodiments, the peptide has a length of 31 residues. In certain embodiments, the peptide has a length of 32 residues. In certain embodiments, the peptide has a length of 33 residues. In certain embodiments, the peptide has a length of 34 residues. In certain embodiments, the peptide has a length of 35 residues. In certain embodiments, the peptide has a length of 36 residues. In certain embodiments, the peptide has a length of 37 residues. In certain embodiments, the peptide has a length of 38 residues. In certain embodiments, the peptide has a length of 39 residues. In certain embodiments, the peptide has a length of 40 residues. In certain embodiments, the peptide has a length of 41 residues. In certain embodiments, the peptide has a length of 42 residues. In certain embodiments, the peptide has a length of 43 residues. In certain embodiments, the peptide has a length of 44 residues. In certain embodiments, the peptide has a length of 45 residues. In certain embodiments, the peptide has a length of 46 residues. In certain embodiments, the peptide has a length of 47 residues. In certain embodiments, the peptide has a length of 48 residues. In certain embodiments, the peptide has a length of 49 residues. In certain embodiments, the peptide has a length of 50 residues.

In certain embodiments, the protein or fragment thereof can be an antibody or an antigen-binding fragment thereof, as disclosed herein.

In certain embodiments, the peptide can be a neoantigen, as disclosed herein.

2. Antibodies or Fragments Thereof

In certain embodiments, a composition analyzed by the methods disclosed herein comprises an antibody or a fragment thereof, e.g., monoclonal antibodies and fragments thereof. For example, but not by way of limitation, the methods of the present disclosure can be used to determine the immunological potential of newly developed and/or identified antibodies or fragments thereof.

Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., Pluckthün, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For a discussion of Fab and F(ab′)₂ fragments comprising salvage receptor binding epitope residues and having increased in vivo half-life, see U.S. Pat. No. 5,869,046. Antibody fragments can be made by various techniques including, but not limited to, proteolytic digestion of an intact antibody as well as production by recombinant host cells (e.g., E. coli or phage), as described herein.

In certain embodiments, a composition analyzed by the methods disclosed herein can be a diabody. Diabodies are antibody fragments comprising two antigen-binding sites that may be bivalent or bispecific. See, for example, EP 404097; WO 1993/01161; Hudson et al., Nat. Med. 9:129-134 (2003); and Hollinger et al., Proc. Natl. Acad. Sci. USA 90: 6444-6448 (1993). Triabodies and tetrabodies, which are also described in Hudson et al., Nat. Med. 9:129-134 (2003) can be analyzed by the disclosed methods.

In certain embodiments, the antibody analyzed by the disclosed methods can be a single-domain antibody. Single-domain antibodies are antibody fragments that comprise all or a portion of the heavy chain variable domain or all or a portion of the light chain variable domain of an antibody. In certain embodiments, a single-domain antibody is a human single-domain antibody (Domantis, Inc., Waltham, Mass.; see, e.g., U.S. Pat. No. 6,248,516 B1). Additional non-limiting examples of single-domain antibodies are disclosed in Iezzi et al., Front Immunol. 9:273 (2018), the contents of which are incorporated by reference herein in their entirety. In certain embodiments, the antibody is a hybribody (Hybrigenics Services, Cambridge, Mass.).

3. Chimeric, Humanized and Human Antibodies

In certain embodiments, a composition analyzed by the methods disclosed herein comprises a chimeric antibody, e.g., a humanized antibody. For example, but not by way of limitation, the presently disclosed methods can be used to identify chimeric versions of an antibody that have a low or lower propensity to elicit productions of ADAs, e.g., compared to the parent antibody or other chimeric versions of the antibody. Alternatively or additionally, methods of the present disclosure can be used to identify chimeric antibodies that have a low propensity to elicit production of ADAs.

Certain chimeric antibodies are described in the art, e.g., in U.S. Pat. No. 4,816,567; and Morrison et al., Proc. Natl. Acad. Sci. USA, 81:6851-6855 (1984)). In certain embodiments, a chimeric antibody includes a non-human variable region (e.g., a variable region derived from a mouse, rat, hamster, rabbit or non-human primate, such as a monkey) and a human constant region. In a further example, a chimeric antibody can be a “class switched” antibody in which the class or subclass has been changed from that of the parent antibody. Chimeric antibodies include antigen-binding fragments thereof.

In certain embodiments, a chimeric antibody can be a humanized antibody. Typically, a non-human antibody is humanized to reduce immunogenicity to humans, while retaining the specificity and affinity of the parental non-human antibody. Generally, a humanized antibody comprises one or more variable domains in which CDRs, e.g., CDRs, (or portions thereof) are derived from a non-human antibody, and FRs (or portions thereof) are derived from human antibody sequences. A humanized antibody optionally will also comprise at least a portion of a human constant region. In certain embodiments, some FR residues in a humanized antibody are substituted with corresponding residues from a non-human antibody (e.g., the antibody from which the CDR residues are derived), e.g., to restore or improve antibody specificity or affinity.

In certain embodiments, a composition analyzed by the methods disclosed herein can be a human antibody. For example, but not by way of limitation, the presently disclosed methods can be used to identify chimeric versions of an antibody that has a low or lower propensity to elicit productions of human antibodies that have a low propensity to elicit production of ADAs.

4. Library-Derived Antibodies

In certain embodiments, a composition analyzed by the methods disclosed herein can comprise an antibody or fragment thereof isolated by screening combinatorial libraries for antibodies with the desired activity or activities. For example, but not by way of limitation, the presently disclosed methods can be used to identify a library-derived antibody that has a low or lower propensity to elicit productions of ADAs, e.g., as compared other library-derived antibodies that possess the desired binding characteristics and/or bind to the same antigen.

Antibodies or antibody fragments isolated from human antibody libraries are considered human antibodies or human antibody fragments herein.

5. Multispecific Antibodies

In certain embodiments, a composition analyzed by the methods disclosed herein can comprise a multispecific antibody, e.g., a bispecific antibody. Multispecific antibodies are monoclonal antibodies that have binding specificities for at least two different epitopes. Bispecific antibodies can be prepared as full-length antibodies or antibody fragments. For example, but not by way of limitation, the presently disclosed methods can be used to identify multispecific antibodies that have a low or lower propensity to elicit productions of ADAs, e.g., compared to other multispecific antibodies that bind the same epitopes. Alternatively, or additionally, methods of the present disclosure can be used to identify multispecific antibodies that have a low propensity to elicit production of ADAs.

In certain embodiments, the presently disclosed methods can be used to identify multispecific antibodies, e.g., bispecific antibodies, that have a low or lower propensity to elicit productions of ADAs, e.g., compared to other antibodies, e.g., monospecific or multispecific antibodies, that bind at least one of the same epitopes as the multispecific antibody.

In certain embodiments, a composition analyzed by the methods disclosed herein can comprise a bispecific antibody that has a binding specificity for T cells. For example, but not limitation, the bispecific antibody can comprise a first antigen-binding domain that binds to T cells, and a second binding specificity for a second epitope, e.g., an epitope that is not present on T cells.

Engineered antibodies with three or more functional antigen binding sites, including “Octopus antibodies,” can also be analyzed by the disclosed methods (see, e.g., US 2006/0025576A1).

6. Immunoconjugates

In certain embodiments, a composition analyzed by the methods disclosed herein can comprise an immunoconjugate, e.g., an immunoconjugate comprising an antibody conjugated to one or more cytotoxic agents, such as chemotherapeutic agents or drugs, growth inhibitory agents, toxins (e.g., protein toxins, enzymatically active toxins of bacterial, fungal, plant or animal origin, or fragments thereof) or radioactive isotopes. For example, an antibody or antigen-binding portion can be functionally linked (e.g., by chemical coupling, genetic fusion, noncovalent association or otherwise) to one or more other binding molecules, such as another antibody, antibody fragment, peptide or binding mimetic.

In certain embodiments, an immunoconjugate is an antibody-drug conjugate (ADC) in which an antibody is conjugated to one or more drugs, including but not limited to a maytansinoid (see U.S. Pat. Nos. 5,208,020, 5,416,064 and European Patent EP 0 425 235); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof (see U.S. Pat. Nos. 5,712,374, 5,714,586, 5,739,116, 5,767,285, 5,770,701, 5,770,710, 5,773,001, and 5,877,296; Hinman et al., Cancer Res. 53:3336-3342 (1993); and Lode et al., Cancer Res. 58:2925-2928 (1998)); an anthracycline such as daunomycin or doxorubicin (see Kratz et al., Current Med. Chem. 13:477-523 (2006); Jeffrey et al., Bioorganic & Med. Chem. Letters 16:358-362 (2006); Torgov et al., Bioconj. Chem. 16:717-721 (2005); Nagy et al., Proc. Natl. Acad. Sci. USA 97:829-834 (2000); Dubowchik et al., Bioorg. & Med. Chem. Letters 12:1529-1532 (2002); King et al., J. Med. Chem. 45:4336-4343 (2002); and U.S. Pat. No. 6,630,579); methotrexate; vindesine; a taxane such as docetaxel, paclitaxel, larotaxel, tesetaxel, and ortataxel; a trichothecene; and CC1065.

In certain embodiments, an immunoconjugate comprises an antibody conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

In certain embodiments, an immunoconjugate comprises an antibody conjugated to a radioactive atom to form a radioconjugate. A variety of radioactive isotopes are available for the production of radioconjugates. Non-limiting examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the radioconjugate is used for detection, it can include a radioactive atom for scintigraphic studies, for example tc99m or I123, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

Conjugates of an antibody and cytotoxic agent can be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker can be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52:127-131 (1992); U.S. Pat. No. 5,208,020) can be used.

In certain embodiments, immunoconjugates include, but are not limited to, such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, Ill., U.S.A).

7. Antibody Variants

In certain embodiments, a composition analyzed by the methods disclosed herein can comprise an antibody variant of a previously disclosed antibody. For example, methods of the present disclosure can be used to identify antibodies that are variants of previously disclosed antibodies that have a lower propensity to elicit productions of ADAs, e.g., than the parent antibody. In certain embodiments, amino acid substitutions can be introduced into an antibody of interest and the antibody variants can be screened for immunogenicity by using the disclosed methods.

In certain embodiments, the antibody variant can be amino acid sequence variants of an antibody, e.g., prepared by introducing appropriate modifications into the nucleotide sequence encoding the antibody or by peptide synthesis. Such modifications include, but are not limited to, deletions from, and/or insertions into and/or substitutions of residues within the amino acid sequences of the antibody. Sites of interest for such variation include, but are not limited to, the CDRs, and FRs. Any combination of deletion, insertion, and substitution can be made to arrive at the final construct, provided that the final antibody, i.e., modified, possesses the desired characteristics, e.g., antigen-binding.

In certain embodiments, an antibody variant can be an antibody that has been altered to increase or decrease the extent to which the antibody is glycosylated. For example, but not by way of limitation, the addition or deletion of glycosylation sites of an antibody can be accomplished by altering the amino acid sequence such that one or more glycosylation sites is created or removed.

In certain embodiments, an antibody analyzed by the methods disclosed herein is an Fc region variant. The Fc region variant can include a human Fc region sequence (e.g., a human IgG1, IgG2, IgG3 or IgG4 Fc region) comprising an amino acid modification (e.g., a substitution) at one or more amino acid positions.

In certain embodiments, an antibody variant can be a cysteine engineered antibody, e.g., “thioMAb,” in which one or more residues of an antibody are substituted with cysteine residues. In certain embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies can be generated as described, e.g., in U.S. Pat. No. 7,521,541.

8. Neoantigens

In certain embodiments, a composition analyzed by the methods disclosed herein can comprise a neoantigen. Neoantigens are antigens that are not associated with normal tissue or organs, and are generally derived from a genetic mutation, e.g., an insertion/deletion, a gene fusion, a frameshift mutation, a single nucleotide mutation, or a combination of the foregoing. In certain embodiments, the neoantigen is a tumor neoantigen (also referred to as a tumor-specific antigen or TSA). As tumor neoantigens are considered “non-self”, they can be processed and displayed via MHC molecules on antigen presenting cells (APCs). Binding of such tumor neoantigens presented on APCs by T cells, e.g., CD8+ and/or CD4+ T cells, is one way in which an immune response can be mounted against the tumor associated with the tumor neoantigen. See, e.g., Jiang et al., J. of Hematology & Oncology 12(93) (2019), the contents of which are incorporated by reference herein.

In certain embodiments, the neoantigen can be analyzed in the context of a complex. For example, but not by way of limitation, the neoantigen can be in a complex with an MHC molecule, e.g., an MHC class I or II molecule. In certain embodiments, the neoantigen can be in a complex with an MHC class II molecule.

A neoantigen for use in the present disclosure can be identified by any method known in the art. For example, but not by way of limitation, a neoantigen can be identified by next-generation sequencing and/or in silico modeling. See, e.g., Garcia-Garijo et al., Front Immunol. 10:1392 (2019), the contents of which are incorporated by reference herein. In certain embodiments, a neoantigen identified by such methods can be analyzed in the presently disclosed methods to determine the propensity of the neoantigen to elicit an immune response specific to the neoantigen.

### IV. Kits

The presently disclosed subject matter further provides kits containing materials useful for performing the methods disclosed herein. In certain embodiments, a kit of the present disclosure includes a container containing lymphocytes and/or a container containing one or more agents for detecting a marker, e.g., CD4, CD134 and/or CD137, described herein. Non-limiting examples of suitable containers include bottles, test tubes, vials and microtiter plates. The containers can be formed from a variety of materials such as glass or plastic.

In certain embodiments, the kit can include one or more containers containing one or more lymphocytes. In certain embodiments, the kit can include at least one container containing PBMCs, lymphocytes, APCs and/or T cells. For example, but not by way of limitation, the kit can include at least one container that includes CD8− T cells, CD4+ T cells and/or CD4+CD8− T cells. In certain embodiments, a kit of the present disclosure includes lymphocytes derived from one or more donors in one or more containers. In certain embodiments, a kit of the present disclosure can further include one or more agents for detecting one or more markers disclosed herein, e.g., CD134 and/or CD137 antibodies.

In certain embodiments, the kit further includes a package insert that provides instructions for using the components provided in the kit. For example, a kit of the present disclosure can include a package insert that provides instructions for using the lymphocytes and/or agents in the disclosed methods.

Alternatively or additionally, the kit can include other materials desirable from a commercial and user standpoint, including other buffers, diluents and filters. In certain embodiments, the kit can include materials for collecting and/or processing blood samples, e.g., to isolate lymphocytes from a sample.

### V. Exemplary Embodiments

A. The presently disclosed subject matter provides a method for determining the propensity of a composition to elicit the production of antibodies specific to said composition relative to a reference propensity, comprising:

(a) culturing lymphocytes in the presence of the composition to generate stimulated lymphocytes;

(b) culturing lymphocytes in the absence of the composition to generate unstimulated lymphocytes;

(c) determining the percentage of the stimulated lymphocytes that are CD4+ and express: (i) CD134; (ii) CD137; or (iii) CD134 and CD137;

(d) determining the percentage of the unstimulated lymphocytes that are CD4+ and express: (i) CD134; (ii) CD137; or (iii) CD134 and CD137; and

(e) calculating a stimulation index value;

wherein when the stimulation index value in (e) is greater than or equal to a reference stimulation index value then the composition has a greater propensity to elicit antibodies specific to said composition and when the stimulation index value in (e) is less than the reference stimulation index value then the composition has a lesser propensity to elicit antibodies specific to said composition.

A1. The foregoing method of A, wherein the reference stimulation index value is from about 1.0 to about 2.0.

A2. The foregoing method of A, wherein the reference stimulation index value is about 1.6 or greater, about 1.7 or greater or about 1.8 or greater.

A3. The foregoing method of any one of A-A2, wherein the stimulation index value is determined by dividing the percentage of stimulated lymphocytes determined in (c) with the percentage of unstimulated lymphocytes determined in (d).

A4. The foregoing method of any one of A-A3, wherein the stimulation index value is determined by outlier sum analysis or determined by linear regression.

A5. The foregoing method of any one of A-A4, wherein the lymphocytes comprise T cells.

A6. The foregoing method of A5, wherein at least 30% of the lymphocytes comprise T cells.

A7. The foregoing method of A5 or A6, wherein the T cells comprise CD8− T cells.

A8. The foregoing method of A7, wherein at least 10% of the T cells comprise CD8− T cells.

A9. The foregoing method of any one of A-A8, wherein the lymphocytes are obtained from a single donor.

A10. The foregoing method of any one of A-A8, wherein the lymphocytes are obtained from about 20 donors to about 50 donors.

A11. The foregoing method of A10, wherein the lymphocytes are obtained from about 35 to about 45 donors.

A12. The foregoing method of A10, wherein the lymphocytes are obtained from at least about 20 donors, at least about 25 donors, at least about 30 donors, at least about 35 donors, at least about 40 donors or at least about 45 donors.

A13. The foregoing method of any one of A-A12, wherein about 1×105 to about 1×107 lymphocytes are cultured with the composition.

A14. The foregoing method of any one of A-A13, wherein the lymphocytes are cultured with about 10 μg/ul to about 1,000 μg/ml of the composition.

A15. The foregoing method of any one of A-A14, wherein the composition comprises a peptide, a polypeptide or a small molecule compound.

A16. The foregoing method of A15, wherein the peptide or polypeptide comprises a neoantigen.

A17. The foregoing method of A15, wherein the polypeptide is an antibody or fragment thereof.

A18. The foregoing method of any one of A17, wherein the antibody is a human, humanized or chimeric antibody.

A19. The foregoing method of any one of A-A14, wherein the composition is an antibody-drug conjugate (ADC).

A20. The foregoing method of any one of A-A19, wherein the lymphocytes are cultured with the composition for about 48 hours or less.

A21. The foregoing method of any one of A-A20, wherein determining the percentage of the stimulated or the unstimulated lymphocytes that are CD4+ and express: (i) CD134; (ii) CD137; or (iii) CD134 and CD137 is performed by flow cytometry.

B. The presently disclosed subject matter provides a method for determining the propensity of a composition to elicit the production of antibodies specific to the composition, comprising:

(a) separately culturing lymphocytes from individual donors in the presence of the composition to generate stimulated lymphocytes;

(b) separately culturing lymphocytes from the individual donors in the absence of the composition to generate unstimulated lymphocytes;

(c) determining the percentage of the stimulated lymphocytes from the individual donors that are CD4+ and express: (i) CD134; (ii) CD137; or (iii) CD134 and CD137;

(d) determining the percentage of the unstimulated lymphocytes from the donors that are CD4+ and express: (i) CD134; (ii) CD137; or (iii) CD134 and CD137;

(e) calculating a stimulation index value for each of the donors; and

(f) calculating a number of reactive lymphocyte donors where the donors' stimulation index value is greater than or equal to a reference value stimulation index value and the number of non-reactive lymphocyte donors where the donors' stimulation index value is less than the reference stimulation index value;

wherein the composition has a high propensity to elicit the production of antibodies specific to the composition if the number of reactive donors is greater than 30% of the total number of donors and the composition has a low propensity to elicit the production of antibodies specific to the composition if the number of reactive donors is less than 20% of the total number of donors.

B1. The foregoing method of B, wherein the reference stimulation index value is from about 1.0 to about 2.0.

B2. The foregoing method of B, wherein the reference stimulation index value is about 1.6 or greater, about 1.7 or greater or about 1.8 or greater.

B3. The foregoing method of any one of B-B2, wherein the stimulation index value is determined by dividing the percentage of stimulated lymphocytes of an individual donor determined in (c) with the percentage of unstimulated lymphocytes of that individual donor determined in (d).

B4. The foregoing method of any one of B-B3, wherein the stimulation index value is determined by outlier sum analysis or determined by linear regression.

B5. The foregoing method of any one of B-B4, wherein the lymphocytes comprise T cells.

B6. The foregoing method of B5, wherein at least 30% of the lymphocytes comprise T cells.

B7. The foregoing method of B5 or B6, wherein the T cells comprise CD8− T cells.

B8. The foregoing method of B7, wherein at least 10% of the T cells comprise CD8− T cells.

B9. The foregoing method of any one of B-B8, wherein the lymphocytes are obtained from about 20 donors to about 50 donors.

B10. The foregoing method of B9, wherein the lymphocytes are obtained from about 35 to about 45 donors.

B11. The foregoing method of B9, wherein the lymphocytes are obtained from at least about 20 donors, at least about 25 donors, at least about 30 donors, at least about 35 donors, at least about 40 donors or at least about 45 donors.

B12. The foregoing method of any one of B-B11, wherein the composition comprises a peptide, a polypeptide or a small molecule compound.

B13. The foregoing method of B12, wherein the polypeptide is an antibody or fragment thereof.

B14. The foregoing method of B13, wherein the antibody is a human, humanized or chimeric antibody.

B15. The foregoing method of B12, wherein the peptide or polypeptide comprises a neoantigen.

B16. The foregoing method of any one of B-B11, wherein the composition is an antibody-drug conjugate (ADC).

B17. The foregoing method of any one of B-B16, wherein the lymphocytes are cultured with the composition for about 48 hours or less.

B18. The foregoing method of any one of B-B17, wherein determining the percentage of the stimulated or the unstimulated lymphocytes that are CD4+ and express: (i) CD134; (ii) CD137; or (iii) CD134 and CD137 is performed by flow cytometry.

C. The presently disclosed subject matter provides a method for determining the propensity of a neoantigen to elicit an immune response specific to said neoantigen relative to a reference antigen, comprising:

(a) culturing lymphocytes in the presence of the neoantigen to generate stimulated lymphocytes;

(b) culturing lymphocytes in the absence of the neoantigen to generate unstimulated lymphocytes;

(c) determining the percentage of the stimulated lymphocytes that are CD4+ and express: (i) CD134; (ii) CD137; or (iii) CD134 and CD137;

(d) determining the percentage of the unstimulated lymphocytes that are CD4+ and express: (i) CD134; (ii) CD137; or (iii) CD134 and CD137; and

(e) calculating a stimulation index value;

wherein when the stimulation index value in (e) is greater than or equal to a reference stimulation index value then the neoantigen has a greater propensity to elicit an immune response specific to said neoantigen and when the stimulation index value in (e) is less than the reference stimulation index value then the neoantigen has a lesser propensity to elicit an immune response specific to said neoantigen.

C1. The foregoing method of C, wherein the neoantigen is present in a complex with an MHC class II molecule.

C2. The foregoing method of C or C1, wherein the reference stimulation index value is from about 1.0 to about 2.0.

C3. The foregoing method of C or C1, wherein the reference stimulation index value is about 1.6 or greater, about 1.7 or greater or about 1.8 or greater.

C4. The foregoing method of any one of C-C3, wherein the stimulation index value is determined by dividing the percentage of stimulated lymphocytes determined in (c) with the percentage of unstimulated lymphocytes determined in (d).

C5. The foregoing method of any one of C-C3, wherein the stimulation index value is determined by outlier sum analysis or determined by linear regression.

C6. The foregoing method of any one of C-05, wherein the lymphocytes comprise T cells.

C7. The foregoing method of C6, wherein at least 30% of the lymphocytes comprise T cells.

C8. The foregoing method of C6 or C7, wherein the T cells comprise CD8− T cells.

C9. The foregoing method of C8, wherein at least 10% of the T cells comprise CD8− T cells.

C10. The foregoing method of any one of C-C9, wherein the lymphocytes are obtained from about 20 donors to about 50 donors.

C11. The foregoing method of C10, wherein the lymphocytes are obtained from about 35 to about 45 donors.

C12. The foregoing method of C10, wherein the lymphocytes are obtained from at least about 20 donors, at least about 25 donors, at least about 30 donors, at least about 35 donors, at least about 40 donors or at least about 45 donors.

C13. The foregoing method of any one of C-C12, wherein the lymphocytes are cultured with the neoantigen for about 48 hours or less.

C14. The foregoing method of any one of C-C13, wherein determining the percentage of the stimulated or the unstimulated lymphocytes that are CD4+ and express: (i) CD134; (ii) CD137; or (iii) CD134 and CD137 is performed by flow cytometry.

D. The presently disclosed subject matter provides a kit for performing any one of the foregoing methods of A-C14.

E. The presently disclosed subject matter provides a method for determining the propensity of a composition to elicit the production of antibodies specific to said composition relative to a reference propensity, comprising:

(a) culturing antigen presenting cells (APCs) in the presence of the composition to generate stimulated APCs;

(b) culturing APCs in the absence of the composition to generate unstimulated APCs;

(c) separately culturing the stimulated APCs with CD4+ lymphocytes and the unstimulated APCs with CD4+ lymphocytes;

(d) determining the percentage of the CD4+ lymphocytes cultured with the stimulated APCs that express: (i) CD134; (ii) CD137; or (iii) CD134 and CD137;

(e) determining the percentage of the CD4+ lymphocytes cultured the unstimulated APCs that express: (i) CD134; (ii) CD137; or (iii) CD134 and CD137; and

(f) calculating a stimulation index value;

wherein when the stimulation index value in (f) is greater than or equal to a reference stimulation index value then the composition has a greater propensity to elicit antibodies specific to said composition and when the stimulation index value in (f) is less than the reference stimulation index value then the composition has a lesser propensity to elicit antibodies specific to said composition.

E1. The foregoing method of E, wherein the reference stimulation index value is from about 1.0 to about 4.0, from about 1.0 to about 3.0 or from about 1.8 to about 3.0.

E2. The foregoing method of E, wherein the reference stimulation index value is about 1.6 or greater, about 1.7 or greater, about 1.8 or greater, about 1.9 or greater, about 2.0 or greater, about 2.1 or greater, about 2.2 or greater, about 2.3 or greater, about 2.4 or greater, about 2.5 or greater, about 2.6 or greater, about 2.7 or greater, about 2.8 or greater, about 2.9 or greater or about 3.0 or greater.

E3. The foregoing method of any one of E-E2, wherein the stimulation index value is determined by dividing the percentage of CD4+ lymphocytes determined in (d) with the percentage of CD4+ lymphocytes determined in (e).

E4. The foregoing method of any one of E-E2, wherein the stimulation index value is determined by outlier sum analysis or determined by linear regression.

E5. The foregoing method of any one of E-E4, wherein the CD4+ lymphocytes comprise CD8− T cells.

E6. The foregoing method of E5, wherein at least 10% of the CD4+ lymphocytes are CD8− T cells.

E7. The foregoing method of any one of E-E6, wherein the APCs are obtained from a single donor.

E8. The foregoing method of any one of E-E6, wherein the APCs are obtained from about 20 donors to about 50 donors.

E9. The foregoing method of E8, wherein the APCs are obtained from about 35 to about 45 donors.

E10. The foregoing method of E8, wherein the APCs are obtained from at least about 20 donors, at least about 25 donors, at least about 30 donors, at least about 35 donors, at least about 40 donors or at least about 45 donors.

E11. The foregoing method of any one of E-E10, wherein about 1×10⁵ to about 1×10⁷ APCs are cultured with the composition.

E12. The foregoing method of any one of E-E11, wherein the APCs are cultured with about 10 μg/ul to about 1,000 μg/ml of the composition.

E13. The foregoing method of any one of E-E12, wherein the composition comprises a peptide, a polypeptide or a small molecule compound.

E14. The foregoing method of E13, wherein the peptide or polypeptide comprises a neoantigen.

E15. The foregoing method of E13, wherein the polypeptide is an antibody or fragment thereof.

E16. The foregoing method of E15, wherein the antibody is a human, humanized or chimeric antibody.

E17. The foregoing method of any one of E-E12, wherein the composition is an antibody-drug conjugate (ADC).

E18. The foregoing method of any one of E-E17, wherein the APCs are cultured with the composition for about 48 hours or less.

E19. The foregoing method of any one of E-E18, wherein determining the percentage of the CD4+ lymphocytes that express: (i) CD134; (ii) CD137; or (iii) CD134 and CD137 is performed by flow cytometry.

E20. The foregoing method of any one of E-E19, wherein determining the percentage of the CD4+ lymphocytes that express: (i) CD134; (ii) CD137; or (iii) CD134 and CD137 is performed by flow cytometry.

F. The presently disclosed subject matter provides a method for determining the propensity of a composition to elicit the production of antibodies specific to the composition, comprising:

(a) separately culturing APCs from individual donors in the presence of the composition to generate stimulated APCs;

(b) separately culturing APCs from the individual donors in the absence of the composition to generate unstimulated APCs;

(c) separately culturing the stimulated APCs with CD4+ lymphocytes and the unstimulated APCs with CD4+ lymphocytes;

(d) determining the percentage of the CD4+ lymphocytes cultured with the stimulated APCs that express: (i) CD134; (ii) CD137; or (iii) CD134 and CD137;

(e) determining the percentage of the CD4+ lymphocytes cultured with the unstimulated APCs that express: (i) CD134; (ii) CD137; or (iii) CD134 and CD137;

(f) calculating a stimulation index value for each of the donors; and

(g) calculating a number of reactive lymphocyte donors where the donors' stimulation index value is greater than or equal to a reference value stimulation index value and the number of non-reactive lymphocyte donors where the donors' stimulation index value is less than the reference stimulation index value;

wherein the composition has a high propensity to elicit the production of antibodies specific to the composition if the number of reactive donors is greater than 30% of the total number of donors and the composition has a low propensity to elicit the production of antibodies specific to the composition if the number of reactive donors is less than 20% of the total number of donors.

F1. The foregoing method of F, wherein the reference stimulation index value is from about 1.0 to about 4.0, from about 1.0 to about 3.0 or from about 1.8 to about 3.0.

F2. The foregoing method of F, wherein the reference stimulation index value is about 1.6 or greater, about 1.7 or greater, about 1.8 or greater, about 1.9 or greater, about 2.0 or greater, about 2.1 or greater, about 2.2 or greater, about 2.3 or greater, about 2.4 or greater, about 2.5 or greater, about 2.6 or greater, about 2.7 or greater, about 2.8 or greater, about 2.9 or greater or about 3.0 or greater.

F3. The foregoing method of any one of F-F2, wherein the stimulation index value is determined by dividing the percentage of CD4+ lymphocytes of an individual donor determined in (d) with the percentage of CD4+ lymphocytes of that individual donor determined in (e).

F4. The foregoing method of any one of F-F2, wherein the stimulation index value is determined by outlier sum analysis or determined by linear regression.

F5. The foregoing method of any of F-F4, wherein the CD4+ lymphocytes comprise CD8− T cells.

F6. The foregoing method of F5, wherein at least 10% of the CD4+ lymphocytes are CD8− T cells.

F7. The foregoing method of any one of F-F6, wherein the APCs are obtained from about 20 donors to about 50 donors.

F8. The foregoing method of F7, wherein the APCs are obtained from about 35 to about 45 donors.

F9. The foregoing method of F7, wherein the APCs are obtained from at least about 20 donors, at least about 25 donors, at least about 30 donors, at least about 35 donors, at least about 40 donors or at least about 45 donors.

F10. The foregoing method of any one of F-F9, wherein the composition comprises a peptide, a polypeptide or a small molecule compound.

F11. The foregoing method of F10, wherein the polypeptide is an antibody or fragment thereof.

F12. The foregoing method of F11, wherein the antibody is a human, humanized or chimeric antibody.

F13. The foregoing method of F10, wherein the peptide or polypeptide comprises a neoantigen.

F14. The foregoing method of any one of F-F9, wherein the composition is an antibody-drug conjugate (ADC).

F15. The foregoing method of any one of F-F14, wherein the APCs are cultured with the composition for about 48 hours or less.

F16. The foregoing method of any one of F-F15, wherein determining the percentage of the CD4+ lymphocytes that express: (i) CD134; (ii) CD137; or (iii) CD134 and CD137 is performed by flow cytometry.

G. The presently disclosed subject matter provides a method for determining the propensity of a neoantigen to elicit an immune response specific to said neoantigen relative to a reference antigen, comprising:

(a) culturing APCs in the presence of the neoantigen to generate stimulated

APCs;

(b) culturing APCs in the absence of the neoantigen to generate unstimulated

APCs;

(c) separately culturing the stimulated APCs with CD4+ lymphocytes and the unstimulated APCs with CD4+ lymphocytes;

(d) determining the percentage of the CD4+ lymphocytes cultured with the stimulated APCs that express: (i) CD134; (ii) CD137; or (iii) CD134 and CD137;

(e) determining the percentage of the CD4+ lymphocytes cultured with the unstimulated APCs that express: (i) CD134; (ii) CD137; or (iii) CD134 and CD137; and

(f) calculating a stimulation index value;

wherein when the stimulation index value in (f) is greater than or equal to a reference stimulation index value then the neoantigen has a greater propensity to elicit an immune response specific to said neoantigen and when the stimulation index value in (0 is less than the reference stimulation index value then the neoantigen has a lesser propensity to elicit an immune response specific to said neoantigen.

G1. The foregoing method of G, wherein the neoantigen is present in a complex with an MHC class II molecule.

G2. The foregoing method of G or G1, wherein the reference stimulation index value is from about 1.0 to about 4.0, from about 1.0 to about 3.0 or from about 1.8 to about 3.0.

G3. The foregoing method of G or G1, wherein the reference stimulation index value is about 1.6 or greater, about 1.7 or greater, about 1.8 or greater, about 1.9 or greater, about 2.0 or greater, about 2.1 or greater, about 2.2 or greater, about 2.3 or greater, about 2.4 or greater, about 2.5 or greater, about 2.6 or greater, about 2.7 or greater, about 2.8 or greater, about 2.9 or greater or about 3.0 or greater.

G4. The foregoing method of any one of G-G3, wherein the stimulation index value is determined by dividing the percentage of CD4+ lymphocytes determined in (d) with the percentage of CD4+ lymphocytes determined in (e).

G5. The foregoing method of any one of G-G3, wherein the stimulation index value is determined by outlier sum analysis or determined by linear regression.

G6. The foregoing method of any one of G-G5, wherein the CD4+ lymphocytes comprise CD8− T cells.

G7. The foregoing method of G6, wherein at least 10% of the CD4+ lymphocytes are CD8− T cells.

G8. The foregoing method of any one of G-G7, wherein the APCs are obtained from about 20 donors to about 50 donors.

G9. The foregoing method of G8, wherein the APCs are obtained from about 35 to about 45 donors.

G10. The foregoing method of G8, wherein the APCs are obtained from at least about 20 donors, at least about 25 donors, at least about 30 donors, at least about 35 donors, at least about 40 donors or at least about 45 donors.

G11. The foregoing method of any one of G-G10, wherein the APCs are cultured with the neoantigen for about 48 hours or less.

G12. The foregoing method of any one of claims G-G11, wherein determining the percentage of the CD4+ lymphocytes that express: (i) CD134; (ii) CD137; or (iii)

CD134 and CD137 is performed by flow cytometry.

H. The presently disclosed subject matter provides a kit for performing any one of the methods of E-G12.

### EXAMPLES

The following examples are merely illustrative of the presently disclosed subject matter and should not be considered as limiting in any way.

### Example 1: T Cell CD4+ Expression Assay

Polypeptide-based therapeutics have the immunogenic potential to elicit the production of ADAs. In particular, such polypeptide-based therapeutics can be taken up and processed by antigen presenting cells such as dendritic cells to present fragments of the polypeptide-based therapeutic in complex with a class II MHC molecule on its surface. T cells subsequently interact with the fragments presented on the surface of the antigen presenting cells to elicit an immune reaction that results in the production of ADAs by B cells.

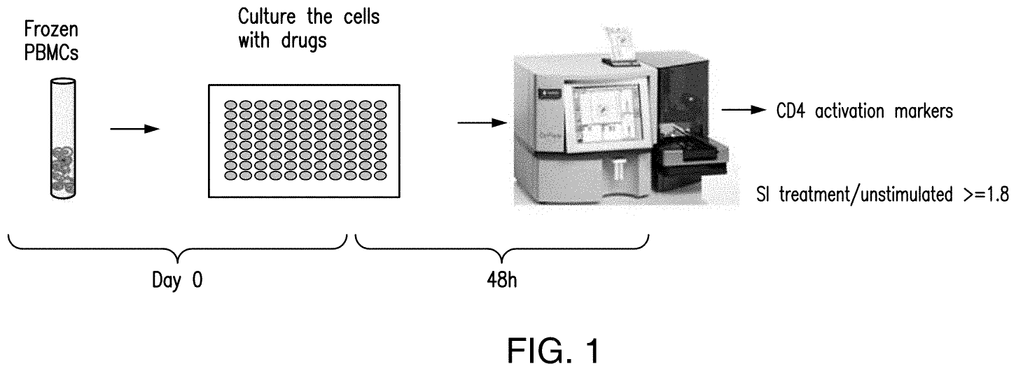

A method for determining the propensity of an antibody to elicit production of ADAs has been developed herein. Such a method can be an extremely valuable tool during drug development as it can be used to predict the immunogenic potential of a newly developed drug in the preclinical stage of development. FIG. 1 provides a schematic of the experimental details of the method. Peripheral (PBMCs) were isolated from naïve healthy donor's blood by density gradient centrifugation using Uni-Sep blood separation tubes. In some experiments, CD8+ cells were depleted using CD8 dynabeads (Thermo Fisher, Waltham, Mass., catalog number: 11147D). It is noted that the assay also yielded good results when the PBMCs were cultured without CD8 depletion. CD8− cells were then cultured with AIM-V medium (Thermo Fisher, Waltham, Mass.) with 10% human AB serum (Sigma Aldrich, catalog number: H3667) at a concentration of 2×10⁶ cells/mL in 24-well plates or 0.2-0.4×10⁶ cells in 96 well plate (Costar, catalog number 3526) and challenged with a final concentration of 100 μg/mL of the tested antibody. All samples are tested in triplicate. For each donor, responses to a negative control, consisting of medium-treated cells (referred to as the unstimulated cells), and positive control with Imject Mariculture KLH (mcKLH) (100 μg/ml) were also included. Cells were placed in a 5% CO₂ incubator at 37° C. for 42-48 hours. Following 42-48 hours, cells were gently resuspended and 200 μl from each 24 well was transferred to a round bottom 96-well plate. CD4 activation was measured by the use of CD4, CD134, CD137 antibodies and live marker. The cells were analyzed by flow cytometry and plots are analyzed using FlowJo FACS analysis software (Tree Star, Inc.; Ashland, Oreg.). For data analysis, the stimulation index (SI) is calculated by dividing the mean and/or max percent of cells that were [live+CD4+CD134+CD137+ and live+CD4+CD134+CD137− and live+CD4+CD134−CD137+] of each treatment by the mean percent of cells that were [live+CD4+CD134+CD137+ and live+CD4+CD134+CD137− and live+CD4+CD134−CD137+] of the medium-only treated well (unstimulated cells) for each treatment.

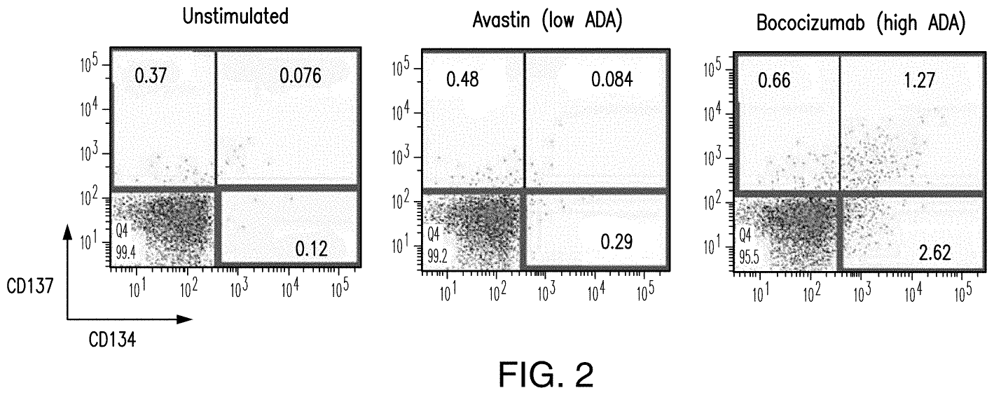

FIG. 2 shows FACS analysis of two different antibodies, AVASTIN® and bococizumab, that have different clinical ADA rates. Bococizumab, which has a high ADA rate, resulted in a higher number of cells that expressed CD4 activation markers compared to AVASTIN®, which has a low ADA rate.

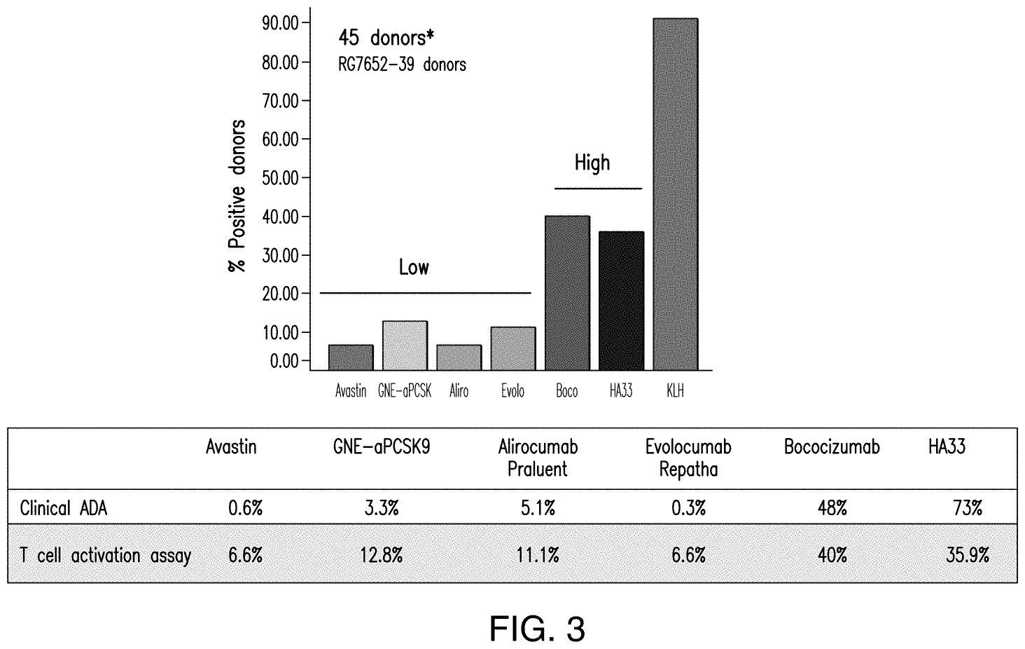

Analysis of six (6) antibodies with different clinical ADA rates confirmed that the results of the disclosed assay correlates with the clinical ADA rate (FIG. 3). Anti-PCSK9 antibodies, AVASTIN®, GNE-αPCSK9, alirocumab (PRALUENT®), evolocumab (REPATHA®), bococizumab and HA33, were analyzed by the method described above. The clinical ADA rates of AVASTIN®, GNE-αPCSK9, alirocumab (PRALUENT®), evolocumab (REPATHA®), bococizumab and HA33A were 0.6%, 3.3%, 5.1%, 0.3%, 48% and 73%, respectively (FIG. 3). As shown in FIG. 3, the number of positive donors for bococizumab or HA33 was significantly higher than any of the other antibodies with low immunogenicity, which relates to the clinical ADA rates observed for the different antibodies.

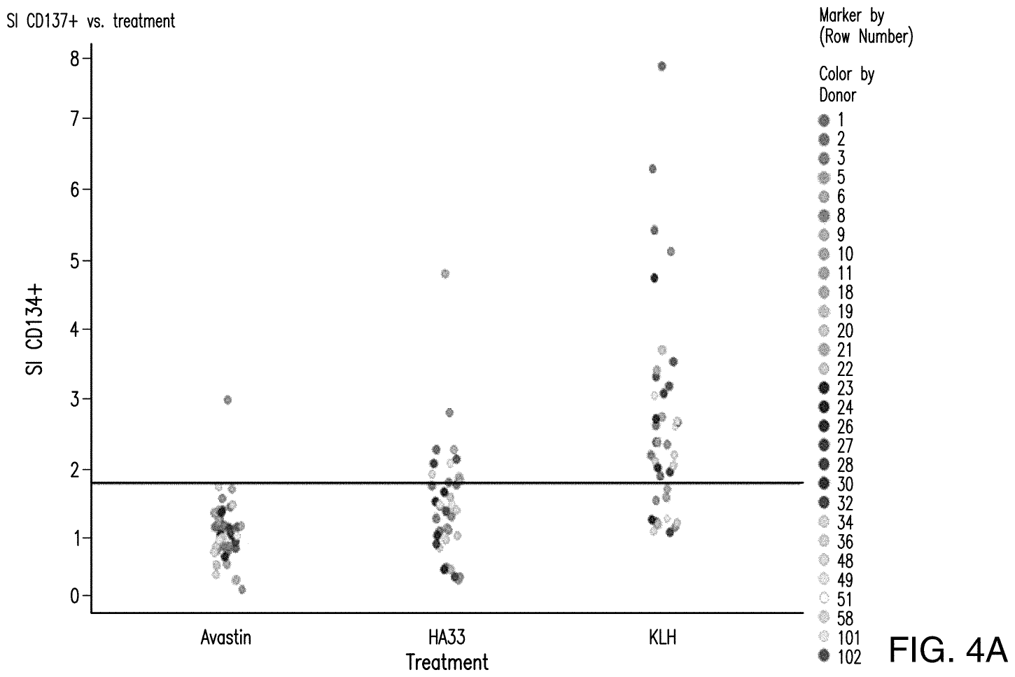

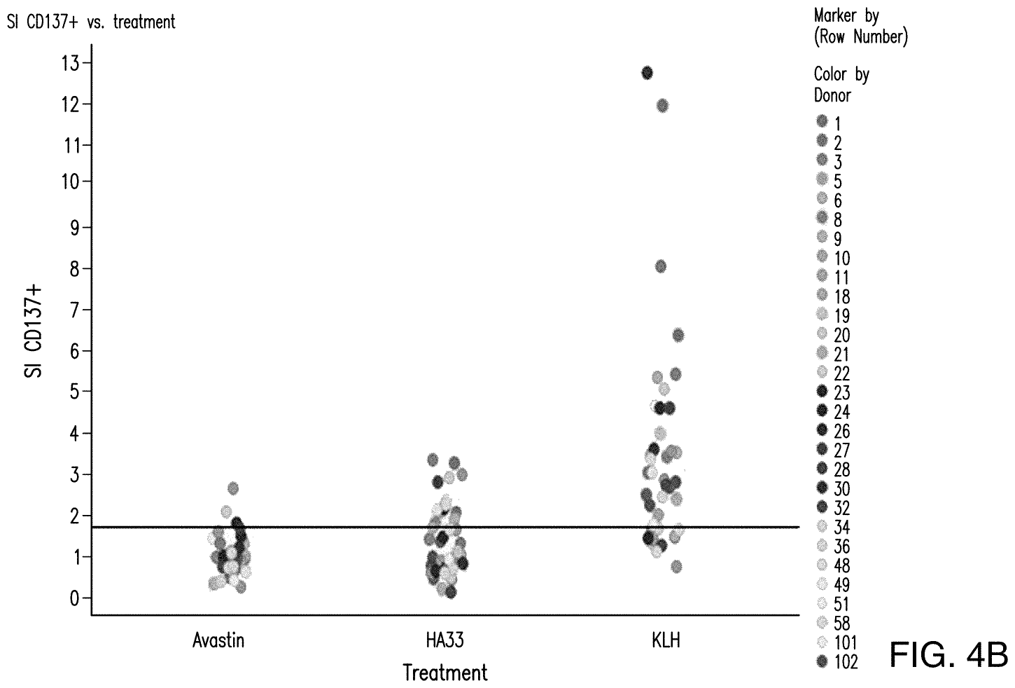

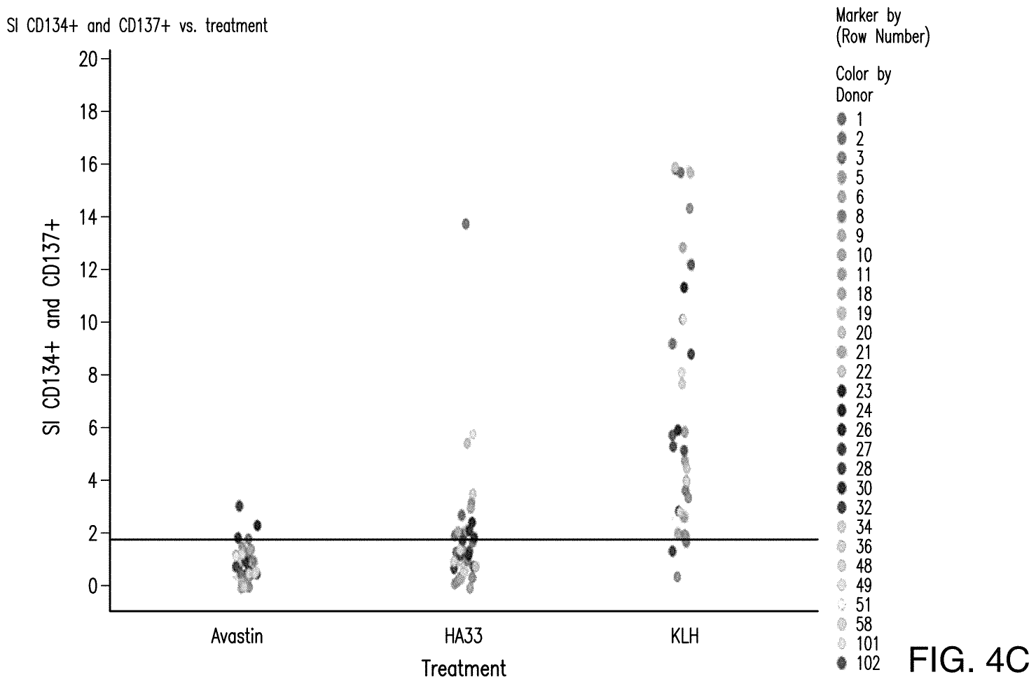

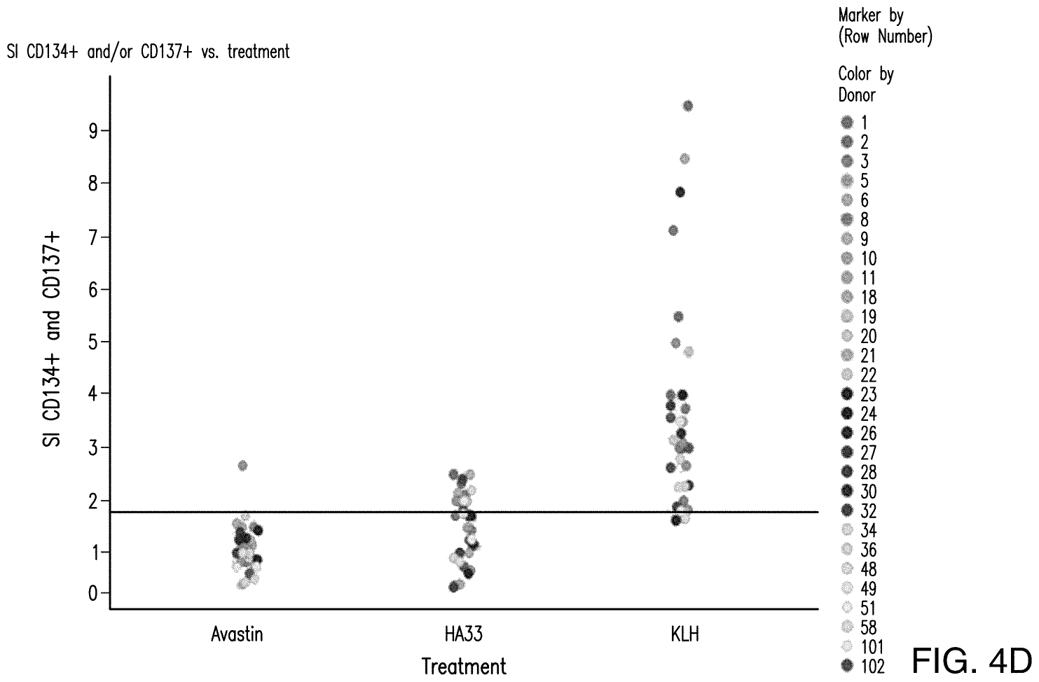

The T cell response of two therapeutics with known ADA in the clinic, HA33 and AVASTIN®, was tested in 40 PBMCs originated from healthy donors. Most of the donors tested positive when stimulated with KLH. In addition, treatment of the cells with AVASTIN® had hardly any effect on either CD134 or CD137 expression; whereas, HA33 treatment showed a marked increase in CD134 (10 donors; FIG. 4A) and CD137 (14 donors; FIG. 4B) and in double positives for CD134 and CD137 (13 donors; FIG. 4C). When CD134 and/or CD137 expression were examined, 14 donors were positive for HA33 and only 1 donor were positive for AVASTIN® (FIG. 4D). Those results correlate with the observed clinical ADA.

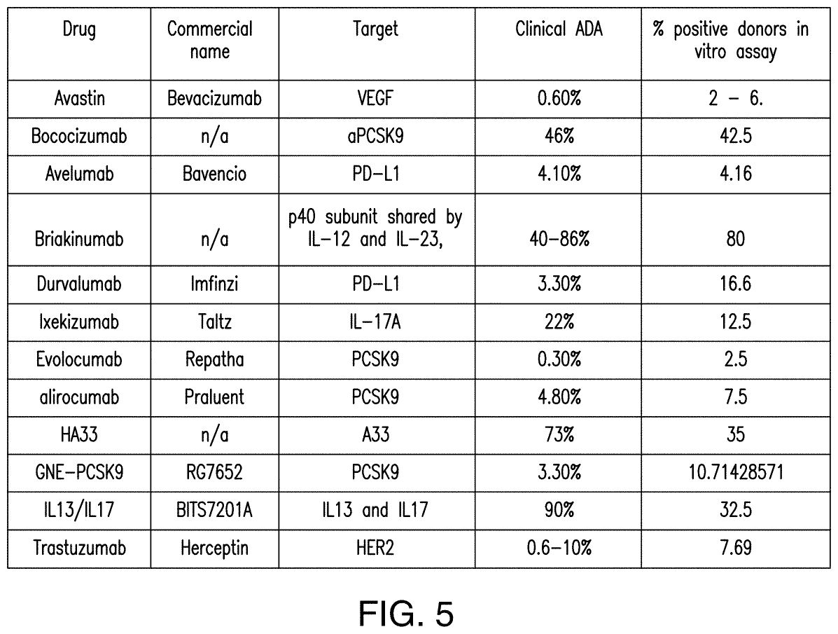

Analysis of additional therapeutics confirm that there is correlation between the predicted immunogenicity and the immunogenicity observed in the clinic. As shown in FIG. 5, those therapeutics with a larger percentage of positive donors in the assay also exhibited a higher clinical ADA rate in the clinic. For example, briakinumab, which resulted in 80% of the donors being positive has a clinical ADA rate of 40-86%; whereas, avelumab (BAVENCIO®) resulted in 4.16% of the donors being positive has a clinical ADA rate of 4.10%.

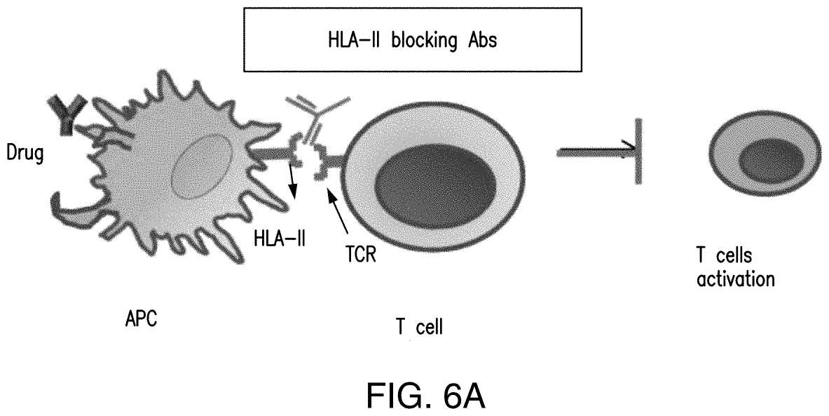

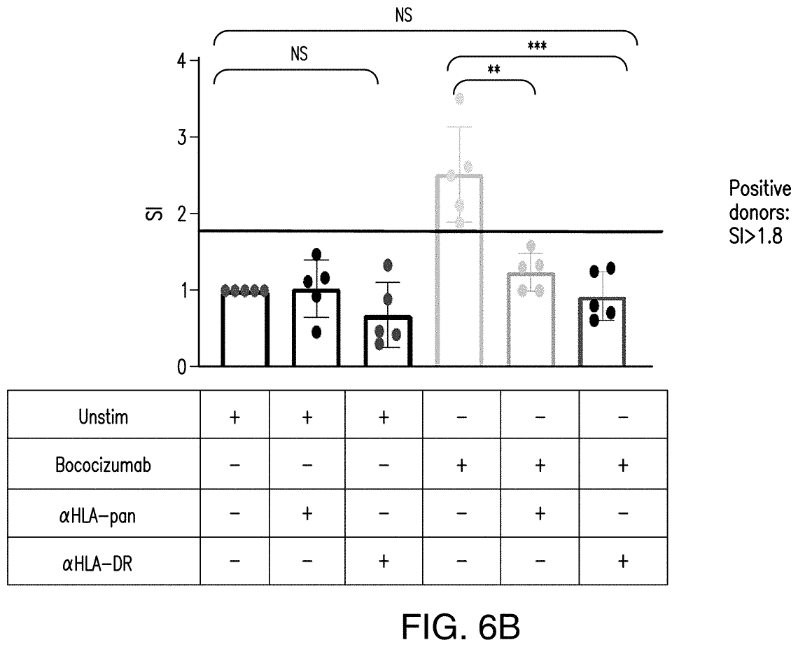

To confirm that the activation of T cells was dependent on presentation of an antigen of the biotherapeutic, the assay was performed in the presence of HLA-DR and HLA-II blocking antibodies (FIG. 6A). As shown in FIG. 6A, antibodies block the interaction of HLA-II to the TCR present on the surface of the T cell thereby blocking the activation of the T cell. As shown in FIG. 6B, blocking HLA-DR and HLA-II proteins reduced the percentage of positive donors indicating that the T cell activation, observed as an increase in CD134 and/or CD137 expression, and determining positive donors is dependent on antigen presentation. These data confirm that CD134 and/or CD137 can be used as markers for activation and that the disclosed assay can predict the immunogenicity of a therapeutic.

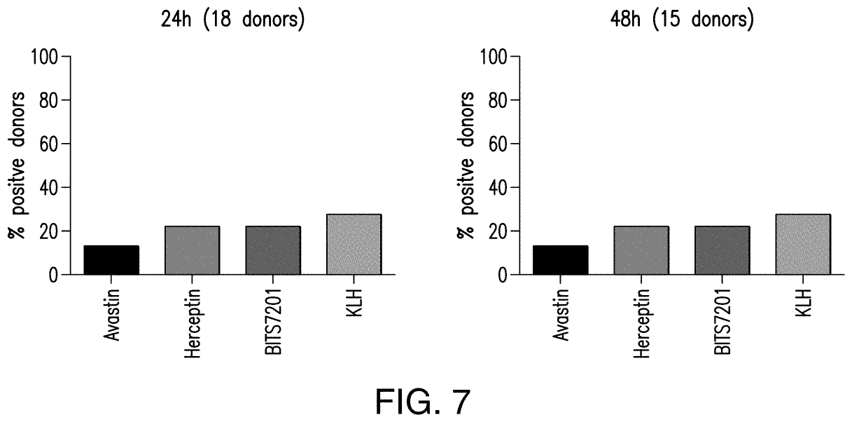

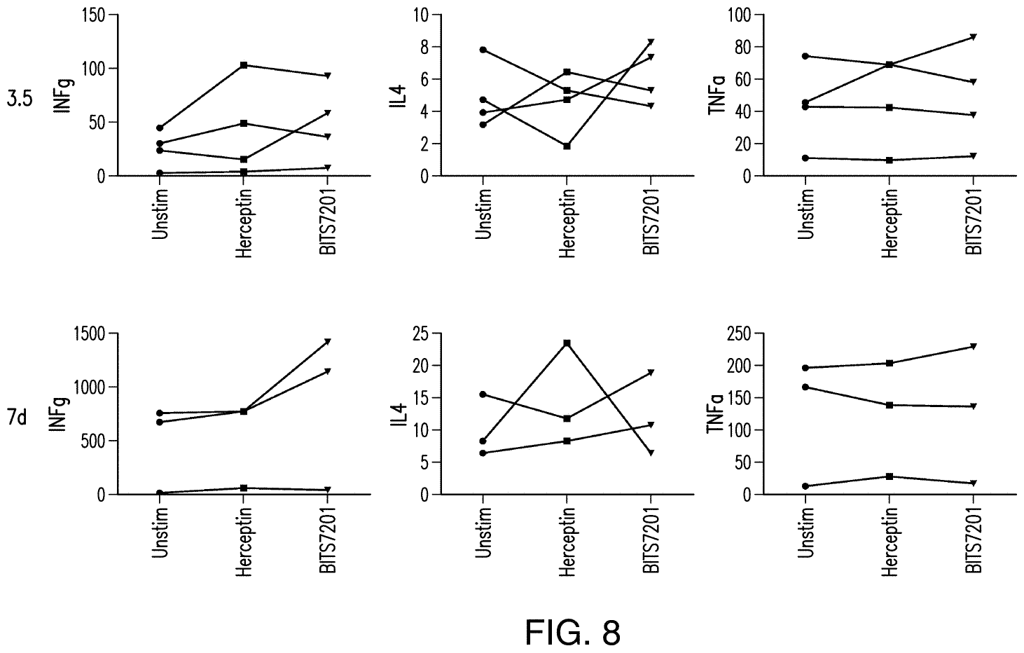

Additional potential markers were assessed to determine if expression of such markers can also be used in this assay. In particular, the secretion and expression of cytokines was analyzed in this assay as potential markers of immunogenicity. As shown in FIG. 7, there was no correlation between the intracellular expression of IL-2 in vitro and clinical immunogenicity. There was also no correlation between the secretion of the cytokines IL-4, TNFα and INFγ in vitro and clinical immunogenicity (FIG. 8).

As shown in Table 1, there are several advantages to the method disclosed herein. Particularly, proliferation assays known in the art take about 20 weeks to perform, require about 2 analysts and costs about $30,000. In contrast, the assay disclosed herein only takes about 2 weeks to perform, requires 1 analyst and costs about $1,000.

TABLE 1

| | Prior Art | |
|---|---|---|
| | Proliferation | Disclosed Assay |
| | Assay to Predict | to Predict |
| | Immunogenicity | Immunogenicity |
| Time | 20 weeks~ | 2 weeks |
| | 4.6 months | |
| Analyst | 2 | 1 |
| Cost | ~$30K | ~$1K |

### Example 2: T Cell Expression Assay for Bispecific Antibodies that Bind T Cells

A method for determining the propensity of an antibody to elicit production of ADAs has been developed herein. A potential challenge of a PBMCs-based assay is interference by the bioactivity of the biotherapeutic of interest, such as immune modulation through direct T cell engagement. To overcome this challenge, a second dendritic cell-T cell assay platform was developed.

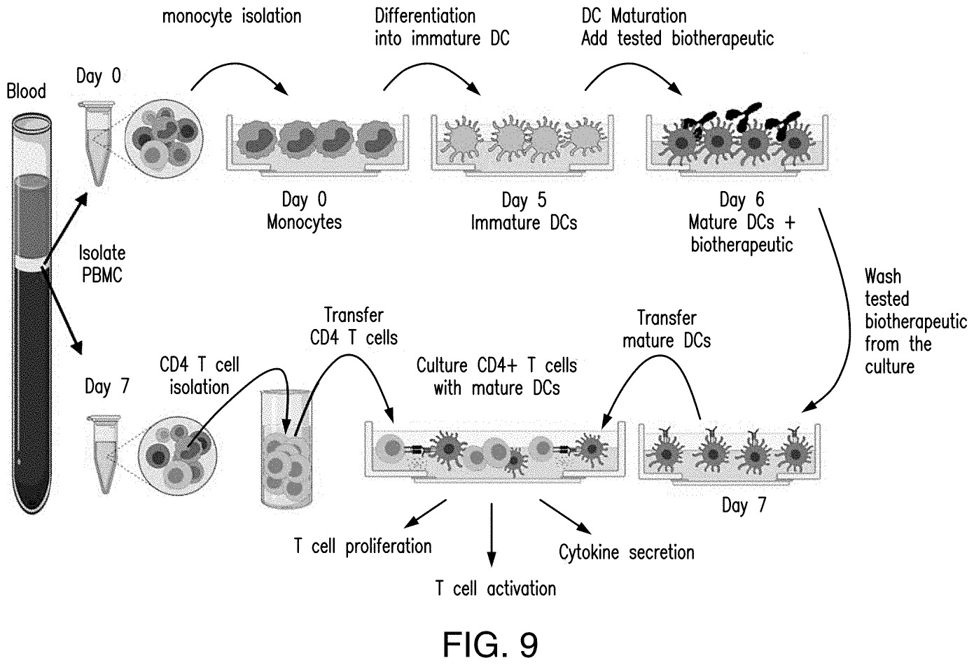

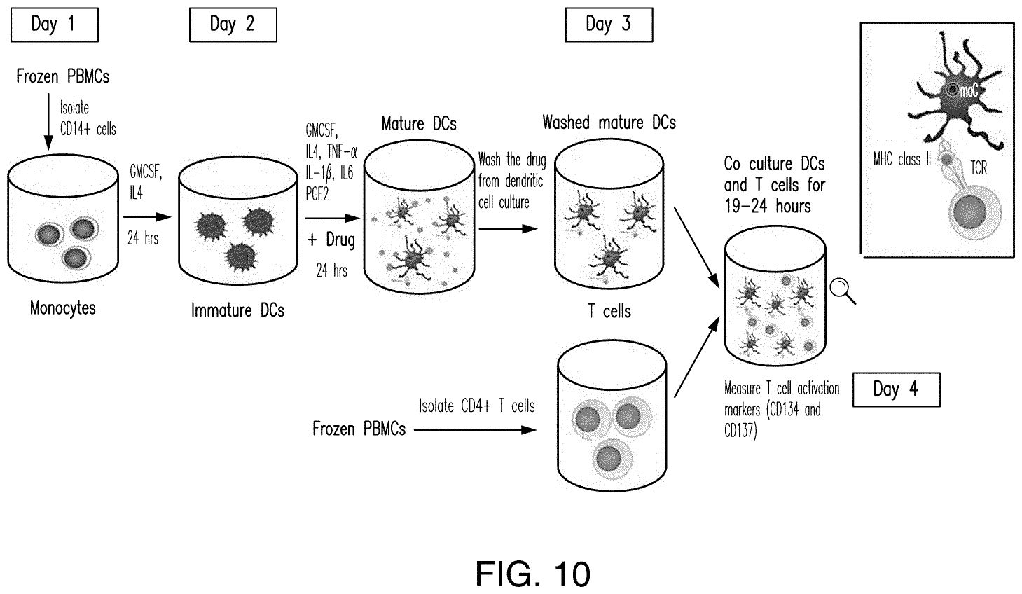

In this assay, PBMC are isolated from naïve healthy donor's blood by density gradient centrifugation using Uni-Sep blood separation tubes and are frozen (max 30×10⁶ PBMCs per tube) in at least 2 tubes. FIG. 9 and FIG. 10 provide schematics of the experimental details of this method, where FIG. 10 provides further details regarding the conditions. On day 1, CD14+ monocytes are isolated from at least 1 tube of PBMCs. Then CD14+ monocytes are cultured at a density of 1.0×10⁶ cells per ml in a 24-well plate with DC Media (RPMI, 1% Non-Essential amino acids, 1% Na-Pyruvate, 1% Kanamycin, 10% AB serum) supplemented with IL4 (17.2 ng/mL) and GM-CSF (66.6 ng/mL) for 24 hours, and placed in a 5% CO₂ incubator. This culturing of CD14+ monocytes enables monocyte differentiation to dendritic cells (DCs). Following 24 hours, the monocyte-derived DCs were washed with sterile PBS and cultured with DC media containing IL4 (17.2 ng/mL), GM-CSF (66.6 ng/mL), TNF-α (5 ng/mL), IL-1β (5 ng/mL), IL-6 (150 ng/mL), PGE2 (1 μg/mL), and 100 μg/ml of the biotherapeutic tested. Cells were placed in a 5% CO₂ incubator. The monocyte-derived DCs were then cultured at a concentration of 0.1 million/mL-200 μL per well (20,000 cells/well in 96 well plate) to enable maturation of the DC for another 24 hours.

At this stage, the mature DCs were exposed to the biotherapeutic for 24 hours to allow antigen uptake and processing and presentation of the antigenic peptides. On day 3, CD4+ cells from the same autologous PBMC population (from an earlier tube) were isolated. In parallel, the mature DCs were washed 3 times with PBS. The CD4+ T cells and matured DCs were co-cultured at a ratio of 5 T cells to 1 DC (200,000 T cells+20,000 DCs). This method allows precise control of the ratio of CD4+ T cells to APCs, which improves assay sensitivity. The ratio of CD4+ T cells and DCs can vary (5:1, 10:1 and 20:1) and can be further modified.

Cells were cultured for at least 19 hours in a 5% CO₂ incubator (for a variable amount of time which could include 24 hours, 48 hours, or 72 hours). All samples were tested in triplicate. For each donor, responses to a negative control consisting of medium-treated cells (referred to as the unstimulated cells) were analyzed. CD4 activation was measured by the use of CD4, CD134, CD137 antibodies and live marker. The cells were analyzed by flow cytometry and plots are analyzed using FlowJo FACS analysis software (Tree Star, Inc.; Ashland, Oreg.). For data analysis, the stimulation index (SI) was calculated by dividing the mean and/or max percent of cells that are [live+CD4+CD134+CD137+, live+CD4+CD134+CD137− and live+CD4+CD134−CD137+] of each treatment by the mean and/or max percent of cells that are [live+CD4+CD134+CD137+ and live+CD4+CD134+CD137− and live+CD4+CD134−CD137+] of the medium-only treated well (unstimulated cells).

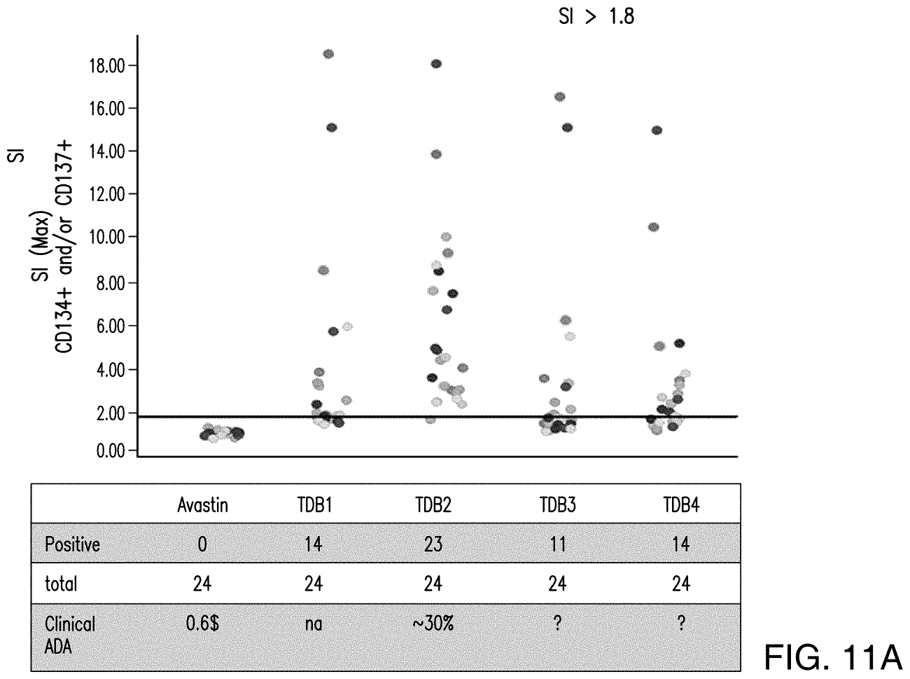

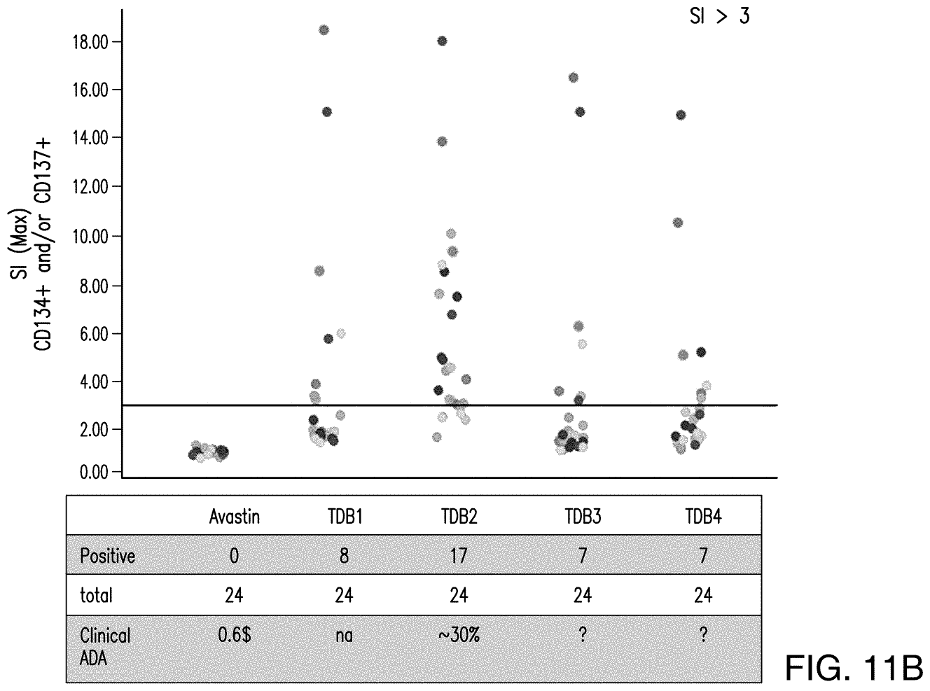

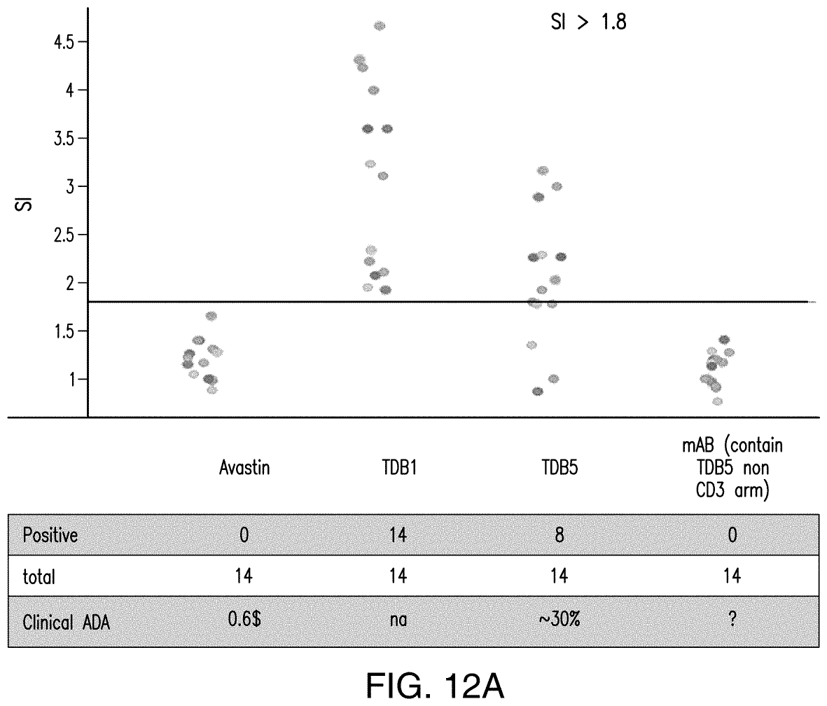

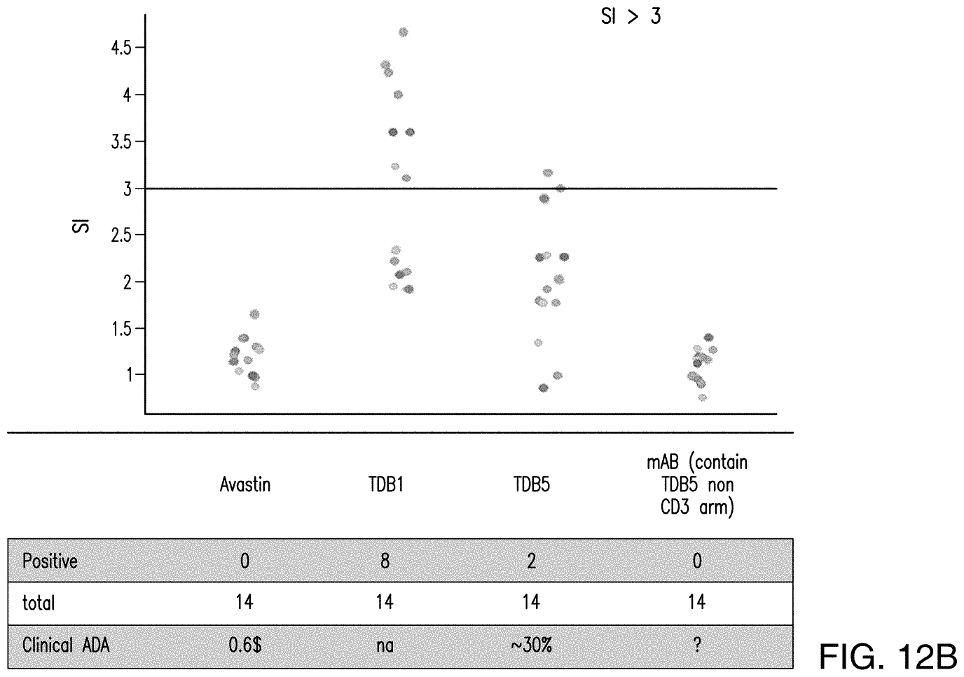

Analysis of five different bispecific antibodies, i.e., TDB1, TDB2, TDB3, TDB4 and TDB5, each of which have an antigen-binding domain that binds to T cells were analyzed by the method described above. As shown in FIGS. 11A, 11B, 12A and 12B, the stimulation indexes of the bispecific antibodies were higher than that of AVASTIN®, which is known to have a low ADA rate. These data suggest that all five bispecific antibodies would be more immunogenic than AVASTIN®.

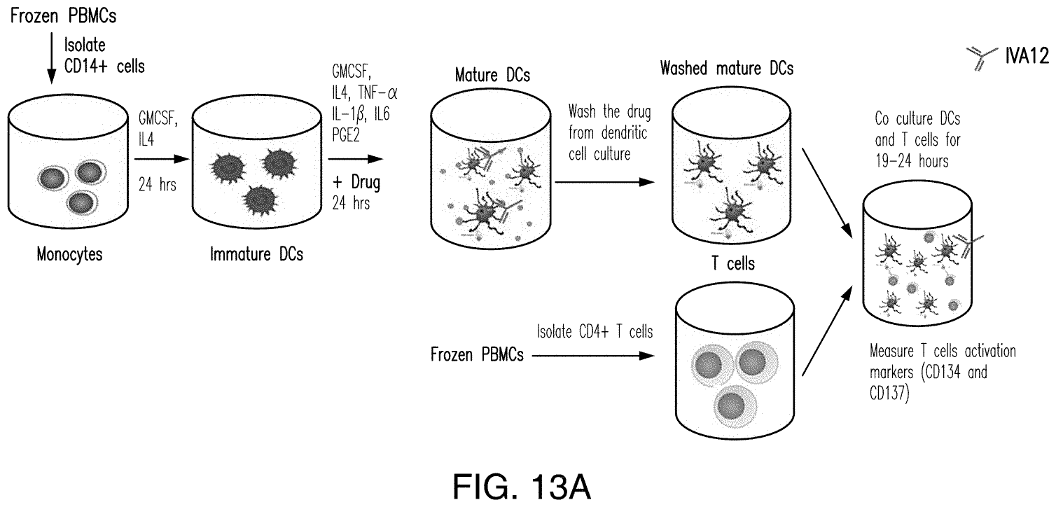

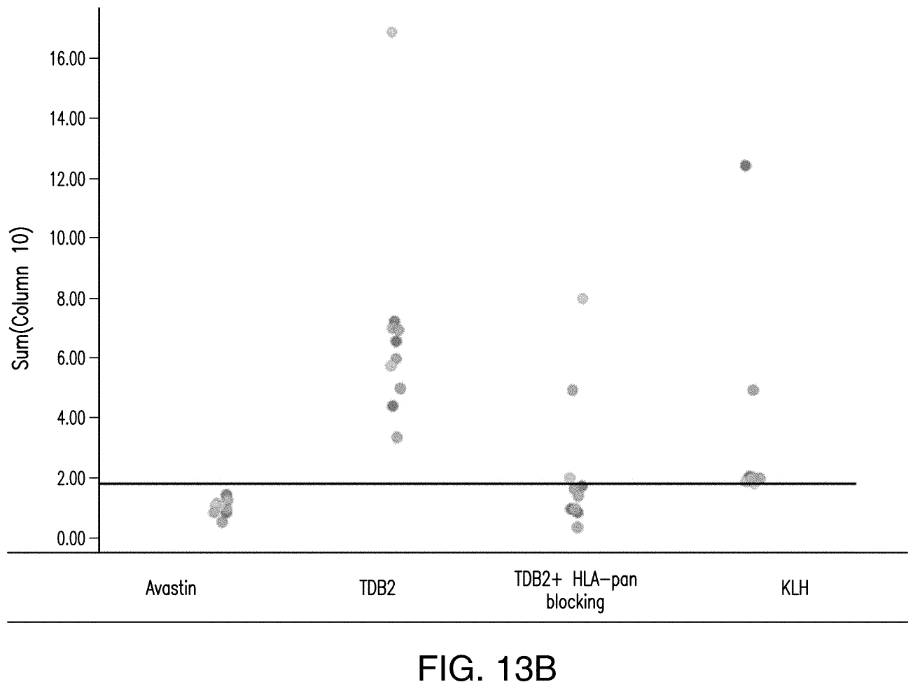

To confirm that the activation of T cells was dependent on presentation of an antigen of the biotherapeutic, the assay was performed in the presence of HLA-II blocking antibodies (FIG. 13A). As shown in FIG. 13B, blocking HLA-II proteins reduced the stimulation index of the bispecific antibody to a value comparable with the reference known to have a low ADA rate.

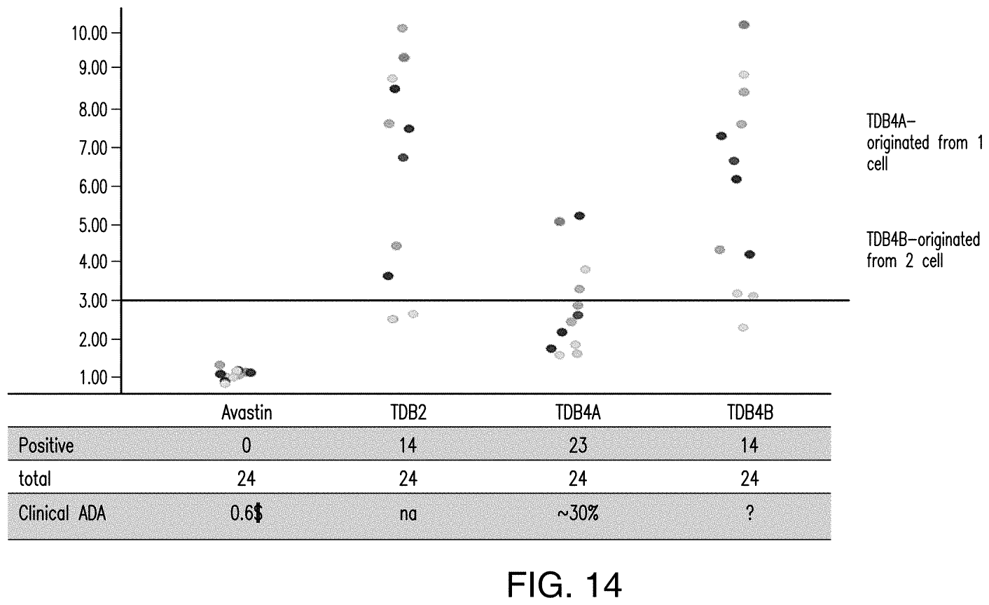

FIG. 14 provides an analysis of a bispecific antibody that has a binding specificity for T cells produced by two different methods. The first method includes the expression of both antigen-binding domains in a single cell to generate the bispecific antibody, which is recited as TDB4A in FIG. 14. The second method includes the expression of each antigen-binding domain in a separate cell and the subsequent isolation and combination of the antigen-binding domains to generate the bispecific antibody, which is recited as TDB4B in FIG. 14. As shown in FIG. 14, TDB4B resulted in a higher stimulation index than TDB4A.

In addition to the various embodiments depicted and claimed, the disclosed subject matter is also directed to other embodiments having other combinations of the features disclosed and claimed herein. As such, the particular features presented herein can be combined with each other in other manners within the scope of the disclosed subject matter such that the disclosed subject matter includes any suitable combination of the features disclosed herein. The foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the compositions and methods of the disclosed subject matter without departing from the spirit or scope of the disclosed subject matter. Thus, it is intended that the disclosed subject matter include modifications and variations that are within the scope of the appended claims and their equivalents.

Various publications, patents and patent applications are cited herein, the contents of which are hereby incorporated by reference in their entireties.

What is claimed is:

1. A method for determining the propensity of a composition to elicit the production of antibodies specific to said composition relative to a reference propensity, comprising:
(a) culturing lymphocytes in the presence of the composition to generate stimulated lymphocytes;
(b) culturing lymphocytes in the absence of the composition to generate unstimulated lymphocytes;
(c) determining the percentage of the stimulated lymphocytes that are CD4+ and express: (i) CD134; (ii) CD137; or (iii) CD134 and CD137;
(d) determining the percentage of the unstimulated lymphocytes that are CD4+ and express: (i) CD134; (ii) CD137; or (iii) CD134 and CD137; and
(e) calculating a stimulation index value;
wherein when the stimulation index value in (e) is greater than or equal to a reference stimulation index value then the composition has a greater propensity to elicit antibodies specific to said composition and when the stimulation index value in (e) is less than the reference stimulation index value then the composition has a lesser propensity to elicit antibodies specific to said composition.

2. The method of claim 1, wherein the reference stimulation index value is from about 1.0 to about 2.0.

3. The method of claim 1, wherein the reference stimulation index value is about 1.6 or greater, about 1.7 or greater or about 1.8 or greater.

4. The method of any one of claims 1-3, wherein the stimulation index value is determined by dividing the percentage of stimulated lymphocytes determined in (c) with the percentage of unstimulated lymphocytes determined in (d).

5. The method of any one of claims 1-3, wherein the stimulation index value is determined by outlier sum analysis or determined by linear regression.

6. The method of any one of claims 1-5, wherein the lymphocytes comprise T cells.

7. The method of claim 6, wherein at least 30% of the lymphocytes comprise T cells.

8. The method of claim 6 or 7, wherein the T cells comprise CD8− T cells.

9. The method of claim 8, wherein at least 10% of the T cells comprise CD8− T cells.

10. The method of any one of claims 1-9, wherein the lymphocytes are obtained from a single donor.

11. The method of any one of claims 1-9, wherein the lymphocytes are obtained from about 20 donors to about 50 donors.

12. The method of claim 11, wherein the lymphocytes are obtained from about 35 to about 45 donors.

13. The method of claim 11, wherein the lymphocytes are obtained from at least about 20 donors, at least about 25 donors, at least about 30 donors, at least about 35 donors, at least about 40 donors or at least about 45 donors.

14. The method of any one of claims 1-13, wherein about 1×10⁵ to about 1×10⁷ lymphocytes are cultured with the composition.

15. The method of any one of claims 1-14, wherein the lymphocytes are cultured with about 10 μg/ul to about 1,000 μg/ml of the composition.

16. The method of any one of claims 1-15, wherein the composition comprises a peptide, a polypeptide or a small molecule compound.

17. The method of claim 16, wherein the peptide or polypeptide comprises a neoantigen.

18. The method of claim 16, wherein the polypeptide is an antibody or fragment thereof.

19. The method of claim 18, wherein the antibody is a human, humanized or chimeric antibody.

20. The method of any one of claims 1-15, wherein the composition is an antibody-drug conjugate (ADC).

21. The method of any one of claims 1-20, wherein the lymphocytes are cultured with the composition for about 48 hours or less.

22. The method of any one of claims 1-21, wherein determining the percentage of the stimulated or the unstimulated lymphocytes that are CD4+ and express: (i) CD134; (ii) CD137; or (iii) CD134 and CD137 is performed by flow cytometry.

23. A method for determining the propensity of a composition to elicit the production of antibodies specific to the composition, comprising:
(a) separately culturing lymphocytes from individual donors in the presence of the composition to generate stimulated lymphocytes;
(b) separately culturing lymphocytes from the individual donors in the absence of the composition to generate unstimulated lymphocytes;
(c) determining the percentage of the stimulated lymphocytes from the individual donors that are CD4+ and express: (i) CD134; (ii) CD137; or (iii) CD134 and CD137;
(d) determining the percentage of the unstimulated lymphocytes from the donors that are CD4+ and express: (i) CD134; (ii) CD137; or (iii) CD134 and CD137;
(e) calculating a stimulation index value for each of the donors; and
(f) calculating a number of reactive lymphocyte donors where the donors' stimulation index value is greater than or equal to a reference value stimulation index value and the number of non-reactive lymphocyte donors where the donors' stimulation index value is less than the reference stimulation index value;
wherein the composition has a high propensity to elicit the production of antibodies specific to the composition if the number of reactive donors is greater than 30% of the total number of donors and the composition has a low propensity to elicit the production of antibodies specific to the composition if the number of reactive donors is less than 20% of the total number of donors.

24. The method of claim 23, wherein the reference stimulation index value is from about 1.0 to about 2.0.

25. The method of claim 23, wherein the reference stimulation index value is about 1.6 or greater, about 1.7 or greater or about 1.8 or greater.

26. The method of any one of claims 23-25, wherein the stimulation index value is determined by dividing the percentage of stimulated lymphocytes of an individual donor determined in (c) with the percentage of unstimulated lymphocytes of that individual donor determined in (d).

27. The method of any one of claims 23-25, wherein the stimulation index value is determined by outlier sum analysis or determined by linear regression.

28. The method of any one of claims 23-27, wherein the lymphocytes comprise T cells.

29. The method of claim 28, wherein at least 30% of the lymphocytes comprise T cells.

30. The method of claim 28 or 29, wherein the T cells comprise CD8− T cells.

31. The method of claim 30, wherein at least 10% of the T cells comprise CD8− T cells.

32. The method of any one of claims 23-31, wherein the lymphocytes are obtained from about 20 donors to about 50 donors.

33. The method of claim 32, wherein the lymphocytes are obtained from about 35 to about 45 donors.

34. The method of claim 32, wherein the lymphocytes are obtained from at least about 20 donors, at least about 25 donors, at least about 30 donors, at least about 35 donors, at least about 40 donors or at least about 45 donors.

35. The method of any one of claims 23-34, wherein the composition comprises a peptide, a polypeptide or a small molecule compound.

36. The method of claim 35, wherein the polypeptide is an antibody or fragment thereof.

37. The method of claim 36, wherein the antibody is a human, humanized or chimeric antibody.

38. The method of claim 35, wherein the peptide or polypeptide comprises a neoantigen.

39. The method of any one of claims 23-34, wherein the composition is an antibody-drug conjugate (ADC).

40. The method of any one of claims 23-39, wherein the lymphocytes are cultured with the composition for about 48 hours or less.

41. The method of any one of claims 23-40, wherein determining the percentage of the stimulated or the unstimulated lymphocytes that are CD4+ and express: (i) CD134; (ii) CD137; or (iii) CD134 and CD137 is performed by flow cytometry.

42. A method for determining the propensity of a neoantigen to elicit an immune response specific to said neoantigen relative to a reference antigen, comprising:
(a) culturing lymphocytes in the presence of the neoantigen to generate stimulated lymphocytes;
(b) culturing lymphocytes in the absence of the neoantigen to generate unstimulated lymphocytes;
(c) determining the percentage of the stimulated lymphocytes that are CD4+ and express: (i) CD134; (ii) CD137; or (iii) CD134 and CD137;
(d) determining the percentage of the unstimulated lymphocytes that are CD4+ and express: (i) CD134; (ii) CD137; or (iii) CD134 and CD137; and
(e) calculating a stimulation index value;
wherein when the stimulation index value in (e) is greater than or equal to a reference stimulation index value then the neoantigen has a greater propensity to elicit an immune response specific to said neoantigen and when the stimulation index value in (e) is less than the reference stimulation index value then the neoantigen has a lesser propensity to elicit an immune response specific to said neoantigen.

43. The method of claim 42, wherein the neoantigen is present in a complex with an MHC class II molecule.

44. The method of claim 42 or 43, wherein the reference stimulation index value is from about 1.0 to about 2.0.

45. The method of claim 42 or 43, wherein the reference stimulation index value is about 1.6 or greater, about 1.7 or greater or about 1.8 or greater.

46. The method of any one of claims 42-45, wherein the stimulation index value is determined by dividing the percentage of stimulated lymphocytes determined in (c) with the percentage of unstimulated lymphocytes determined in (d).

47. The method of any one of claims 42-45, wherein the stimulation index value is determined by outlier sum analysis or determined by linear regression.

48. The method of any one of claims 42-47, wherein the lymphocytes comprise T cells.

49. The method of claim 48, wherein at least 30% of the lymphocytes comprise T cells.

50. The method of claim 48 or 49, wherein the T cells comprise CD8− T cells.

51. The method of claim 50, wherein at least 10% of the T cells comprise CD8− T cells.

52. The method of any one of claims 42-51, wherein the lymphocytes are obtained from about 20 donors to about 50 donors.

53. The method of claim 52, wherein the lymphocytes are obtained from about 35 to about 45 donors.

54. The method of claim 52, wherein the lymphocytes are obtained from at least about 20 donors, at least about 25 donors, at least about 30 donors, at least about 35 donors, at least about 40 donors or at least about 45 donors.

55. The method of any one of claims 42-54, wherein the lymphocytes are cultured with the neoantigen for about 48 hours or less.

56. The method of any one of claims 42-55, wherein determining the percentage of the stimulated or the unstimulated lymphocytes that are CD4+ and express: (i) CD134; (ii) CD137; or (iii) CD134 and CD137 is performed by flow cytometry.

57. A kit for performing any one of the methods of claims 1-56.

58. A method for determining the propensity of a composition to elicit the production of antibodies specific to said composition relative to a reference propensity, comprising:
(a) culturing antigen presenting cells (APCs) in the presence of the composition to generate stimulated APCs;
(b) culturing APCs in the absence of the composition to generate unstimulated APCs;
(c) separately culturing the stimulated APCs with CD4+ lymphocytes and the unstimulated APCs with CD4+ lymphocytes;
(d) determining the percentage of the CD4+ lymphocytes cultured with the stimulated APCs that express: (i) CD134; (ii) CD137; or (iii) CD134 and CD137;
(e) determining the percentage of the CD4+ lymphocytes cultured the unstimulated APCs that express: (i) CD134; (ii) CD137; or (iii) CD134 and CD137; and
(f) calculating a stimulation index value;
wherein when the stimulation index value in (f) is greater than or equal to a reference stimulation index value then the composition has a greater propensity to elicit antibodies specific to said composition and when the stimulation index value in (f) is less than the reference stimulation index value then the composition has a lesser propensity to elicit antibodies specific to said composition.

59. The method of claim 58, wherein the reference stimulation index value is from about 1.0 to about 4.0, from about 1.0 to about 3.0 or from about 1.8 to about 3.0.

60. The method of claim 58, wherein the reference stimulation index value is about 1.6 or greater, about 1.7 or greater, about 1.8 or greater, about 1.9 or greater, about 2.0 or greater, about 2.1 or greater, about 2.2 or greater, about 2.3 or greater, about 2.4 or greater, about 2.5 or greater, about 2.6 or greater, about 2.7 or greater, about 2.8 or greater, about 2.9 or greater or about 3.0 or greater.

61. The method of any one of claims 58-60, wherein the stimulation index value is determined by dividing the percentage of CD4+ lymphocytes determined in (d) with the percentage of CD4+ lymphocytes determined in (e).

62. The method of any one of claims 58-60, wherein the stimulation index value is determined by outlier sum analysis or determined by linear regression.

63. The method of any one of claims 58-62, wherein the CD4+ lymphocytes comprise CD8− T cells.

64. The method of claim 63, wherein at least 10% of the CD4+ lymphocytes are CD8− T cells.

65. The method of any one of claims 58-64, wherein the APCs are obtained from a single donor.

66. The method of any one of claims 58-64, wherein the APCs are obtained from about 20 donors to about 50 donors.

67. The method of claim 66, wherein the APCs are obtained from about 35 to about 45 donors.

68. The method of claim 66, wherein the APCs are obtained from at least about 20 donors, at least about 25 donors, at least about 30 donors, at least about 35 donors, at least about 40 donors or at least about 45 donors.

69. The method of any one of claims 58-68, wherein about 1×10⁵ to about 1×10⁷ APCs are cultured with the composition.

70. The method of any one of claims 58-69, wherein the APCs are cultured with about 10 μg/ul to about 1,000 μg/ml of the composition.

71. The method of any one of claims 58-70, wherein the composition comprises a peptide, a polypeptide or a small molecule compound.

72. The method of claim 71, wherein the peptide or polypeptide comprises a neoantigen.

73. The method of claim 71, wherein the polypeptide is an antibody or fragment thereof.

74. The method of claim 73, wherein the antibody is a human, humanized or chimeric antibody.

75. The method of any one of claims 58-70, wherein the composition is an antibody-drug conjugate (ADC).

76. The method of any one of claims 58-75, wherein the APCs are cultured with the composition for about 48 hours or less.

77. The method of any one of claims 58-76, wherein determining the percentage of the CD4+ lymphocytes that express: (i) CD134; (ii) CD137; or (iii) CD134 and CD137 is performed by flow cytometry.

78. A method for determining the propensity of a composition to elicit the production of antibodies specific to the composition, comprising:
(a) separately culturing APCs from individual donors in the presence of the composition to generate stimulated APCs;
(b) separately culturing APCs from the individual donors in the absence of the composition to generate unstimulated APCs;
(c) separately culturing the stimulated APCs with CD4+ lymphocytes and the unstimulated APCs with CD4+ lymphocytes;
(d) determining the percentage of the CD4+ lymphocytes cultured with the stimulated APCs that express: (i) CD134; (ii) CD137; or (iii) CD134 and CD137;
(e) determining the percentage of the CD4+ lymphocytes cultured with the unstimulated APCs that express: (i) CD134; (ii) CD137; or (iii) CD134 and CD137;
(f) calculating a stimulation index value for each of the donors; and
(g) calculating a number of reactive lymphocyte donors where the donors' stimulation index value is greater than or equal to a reference value stimulation index value and the number of non-reactive lymphocyte donors where the donors' stimulation index value is less than the reference stimulation index value;
wherein the composition has a high propensity to elicit the production of antibodies specific to the composition if the number of reactive donors is greater than 30% of the total number of donors and the composition has a low propensity to elicit the production of antibodies specific to the composition if the number of reactive donors is less than 20% of the total number of donors.

79. The method of claim 78, wherein the reference stimulation index value is from about 1.0 to about 4.0, from about 1.0 to about 3.0 or from about 1.8 to about 3.0.

80. The method of claim 78, wherein the reference stimulation index value is about 1.6 or greater, about 1.7 or greater, about 1.8 or greater, about 1.9 or greater, about 2.0 or greater, about 2.1 or greater, about 2.2 or greater, about 2.3 or greater, about 2.4 or greater, about 2.5 or greater, about 2.6 or greater, about 2.7 or greater, about 2.8 or greater, about 2.9 or greater or about 3.0 or greater.

81. The method of any one of claims 78-80, wherein the stimulation index value is determined by dividing the percentage of CD4+ lymphocytes of an individual donor determined in (d) with the percentage of CD4+ lymphocytes of that individual donor determined in (e).

82. The method of any one of claims 78-80, wherein the stimulation index value is determined by outlier sum analysis or determined by linear regression.

83. The method of any of claims 78-82, wherein the CD4+ lymphocytes comprise CD8− T cells.

84. The method of claim 83, wherein at least 10% of the CD4+ lymphocytes are CD8− T cells.

85. The method of any one of claims 78-84, wherein the APCs are obtained from about 20 donors to about 50 donors.

86. The method of claim 85, wherein the APCs are obtained from about 35 to about 45 donors.

87. The method of claim 85, wherein the APCs are obtained from at least about 20 donors, at least about 25 donors, at least about 30 donors, at least about 35 donors, at least about 40 donors or at least about 45 donors.

88. The method of any one of claims 78-87, wherein the composition comprises a peptide, a polypeptide or a small molecule compound.

89. The method of claim 88, wherein the polypeptide is an antibody or fragment thereof.

90. The method of claim 89, wherein the antibody is a human, humanized or chimeric antibody.

91. The method of claim 88, wherein the peptide or polypeptide comprises a neoantigen.

92. The method of any one of claims 78-87, wherein the composition is an antibody-drug conjugate (ADC).

93. The method of any one of claims 78-92, wherein the APCs are cultured with the composition for about 48 hours or less.

94. The method of any one of claims 78-93, wherein determining the percentage of the CD4+ lymphocytes that express: (i) CD134; (ii) CD137; or (iii) CD134 and CD137 is performed by flow cytometry.

95. A method for determining the propensity of a neoantigen to elicit an immune response specific to said neoantigen relative to a reference antigen, comprising:
(a) culturing APCs in the presence of the neoantigen to generate stimulated APCs;
(b) culturing APCs in the absence of the neoantigen to generate unstimulated APCs;
(c) separately culturing the stimulated APCs with CD4+ lymphocytes and the unstimulated APCs with CD4+ lymphocytes;
(d) determining the percentage of the CD4+ lymphocytes cultured with the stimulated APCs that express: (i) CD134; (ii) CD137; or (iii) CD134 and CD137;
(e) determining the percentage of the CD4+ lymphocytes cultured with the unstimulated APCs that express: (i) CD134; (ii) CD137; or (iii) CD134 and CD137; and
(f) calculating a stimulation index value;
wherein when the stimulation index value in (f) is greater than or equal to a reference stimulation index value then the neoantigen has a greater propensity to elicit an immune response specific to said neoantigen and when the stimulation index value in (f) is less than the reference stimulation index value then the neoantigen has a lesser propensity to elicit an immune response specific to said neoantigen.

96. The method of claim 95, wherein the neoantigen is present in a complex with an MHC class II molecule.

97. The method of claim 95 or 96, wherein the reference stimulation index value is from about 1.0 to about 4.0, from about 1.0 to about 3.0 or from about 1.8 to about 3.0.

98. The method of claim 95 or 96, wherein the reference stimulation index value is about 1.6 or greater, about 1.7 or greater, about 1.8 or greater, about 1.9 or greater, about 2.0 or greater, about 2.1 or greater, about 2.2 or greater, about 2.3 or greater, about 2.4 or greater, about 2.5 or greater, about 2.6 or greater, about 2.7 or greater, about 2.8 or greater, about 2.9 or greater or about 3.0 or greater.

99. The method of any one of claims 95-98, wherein the stimulation index value is determined by dividing the percentage of CD4+ lymphocytes determined in (d) with the percentage of CD4+ lymphocytes determined in (e).

100. The method of any one of claims 95-98, wherein the stimulation index value is determined by outlier sum analysis or determined by linear regression.

101. The method of any one of claims 95-100, wherein the CD4+ lymphocytes comprise CD8− T cells.

102. The method of claim 101, wherein at least 10% of the CD4+ lymphocytes are CD8− T cells.

103. The method of any one of claims 95-102, wherein the APCs are obtained from about 20 donors to about 50 donors.

104. The method of claim 103, wherein the APCs are obtained from about 35 to about 45 donors.

105. The method of claim 103, wherein the APCs are obtained from at least about 20 donors, at least about 25 donors, at least about 30 donors, at least about 35 donors, at least about 40 donors or at least about 45 donors.

106. The method of any one of claims 95-105, wherein the APCs are cultured with the neoantigen for about 48 hours or less.

107. The method of any one of claims 95-106, wherein determining the percentage of the CD4+ lymphocytes that express: (i) CD134; (ii) CD137; or (iii) CD134 and CD137 is performed by flow cytometry.

108. A kit for performing any one of the methods of claims 58-107.
